(19) United States
(12) Patent Application Publication
(10) Pub. No.: US 2019/0206571 A1
(43) Pub. Date: Jul. 4, 2019

(54) SYSTEMS AND METHODS FOR RECOMMENDING DISEASE-RELATED CONTENT BASED ON A DISEASE ASSOCIATED WITH A USER

(71) Applicant: ROVI Guides, Inc., San Jose, CA (US)

(72) Inventors: Adam Bates, Los Gatos, CA (US); Jesse F. Patterson, Pacifica, CA (US); Mark K. Berner, Santa Clara, CA (US); Eric Dorsey, Palo Alto, CA (US); David W. Chamberlin, Pacifica, CA (US); Paul Stevens, Felton, CA (US); Herbert A. Waterman, Merced, CA (US)

(21) Appl. No.: 15/857,072

(22) Filed: Dec. 28, 2017

(51) Int. Cl. G16H 50/70; G16H 10/60; G06F 17/30; G06N 3/00

(52) U.S. Cl. CPC G16H 50/70; G16H 10/60; G06N 3/006; G06F 17/3053; G06F 17/30554

(57) ABSTRACT

Systems and methods are disclosed herein for recommending disease related content based on a disease associated with a user. A user may request his or her genetic testing data. That genetic testing data may then be used to determine which genetic disease the user may possesses. Furthermore, the genetic testing data may indicate genetically-linked offspring of the user that may also be affected by a mutation that can lead to a genetic disease. The media guidance application may then recommend disease-related content to both the user and offspring.

### BACKGROUND

It has become increasingly desirable for parents to perform take home genetic tests, such as 23andMe genetic tests, in order to learn about the diseases that may affect the parent or the parent's offspring. Most parents have limited knowledge of how to understand the results of a genetic test, causing parents to reach out to expensive genetic consultants to truly understand their testing results.

Additionally, parents may find it difficult to share the results of the genetic tests with their offspring, who may be affected. Traditional systems have no mechanism to enable a parent to leverage documentaries, articles, shows, movies, etc., that could aid in teaching his/her offspring more about the disease.

### SUMMARY

Accordingly, systems and methods are described herein for a media guidance application that recommends content, featuring a genetically identifiable disease, based on a positive determination of a genetic marker that suggests a likelihood of having a certain disease. For example, if a first user who has offspring, such as a father in a family with two children, has genetic variations that indicate a high likelihood of developing Alzheimer's disease, a neurological disease, later in life; the systems and methods provided herein selectively recommend content to users with a genetic link to the father, such as the father's son, about Alzheimer's disease. Thus, the father, or any first user can have assistance in communicating with family members about sensitive health topics and the risks and realities of certain genetic diseases.

For example, the media guidance application may store various family member profiles in the same location in memory, such as a four-member family consisting of a father, mother, daughter, and son. The media guidance application may selectively access a certain family member's genetic testing data in order to identify genetic risks associated with each individual in the family. The media guidance application may identify a genetic link between the father and the son, for example. The media guidance application may then identify a certain genetically linked disease between the father and the son, such as Alzheimer's disease, and present disease-related content to the son, thus alleviating some of the father's stress in communicating sensitive information to the son.

In some aspects, the media guidance application may request access to a first user's genomic sequencing testing data. For example, the media guidance application may identify a first user from a variety of users stored in local memory on a profile database. The media guidance application may identify the first user as the father in a family of four people with a mother, a daughter, and a son. Furthermore, the media guidance application may request access to the father's genomic sequencing testing data. In some embodiments, the media guidance application may retrieve a genetic sample from the first user. For example, the media guidance application may access a database from an entity such as 23andMe, which is a genetic testing company that provides a direct-to-consumer genetic testing method. The media guidance application may retrieve the father's genetic testing results and analysis from the previously mentioned 23andMe database.

The media guidance application may, in response to requesting the first user's genomic sequencing testing data, retrieve from a profile associated with the first user, the first user's genomic sequencing testing data. For example, as previously introduced, the media guidance application may access user profiles stored in memory, one of which is associated with a first user, the father. In a further example, the media guidance application may request the father's 23andMe genomic sequencing test results and analysis from the 23andMe database. For example, the media guidance application may access the father's 23andMe results and analysis and use the results and analysis to identify variations in the father's chromosomes, genes, and proteins.

The media guidance application may search, based on the retrieved first user's genomic sequencing testing data, a database that associates genomic sequencing data with diseases to identify a disease associated with the first user. For example, the media guidance application may access and search a 23andMe database that associates the father's genomic testing results with various diseases. Furthermore, the media guidance application may determine, from the directory of diseases, that the father has a genetic variation associated with Alzheimer's disease. In some embodiments, the media guidance application may identify a genetic-related disease, which is a disorder or condition caused by abnormalities in a person's genome. For example, the media guidance application may search a database of genomic information that has various diseases associated with abnormalities in an individual's genome. In some embodiments, the media guidance application may identify a genomic abnormality, wherein a genome further comprises genetic material of an organism, such as a human, that contains information about that organism's traits and characteristics, including the organism's likelihood of having a certain disease.

In some embodiments, the media guidance application may present results to the first user based on the analysis, which indicates at least one of a likelihood that the first user is a carrier of a particular genetic mutation, the first user is associated with an increased risk of developing a disease, such as cancer, in the future, and a need for further testing. For example, the media guidance application may present results to the first user, the father, indicating the likelihood that he is a carrier for a certain disease. In some embodiments, the media guidance application may generate content for presentation to the first user based on the genetic data of the first user. For example, the media guidance application may search and retrieve various media content to identify Alzheimer's-related content for the father. Furthermore, the media guidance application may present the Alzheimer's-related content to the father.

In some embodiments, the media guidance application may generate a playlist of content that includes a first media asset corresponding to a background of the first user based on the genetic data and a second media asset corresponding to the disease. For example, based on the results and analysis of the 23andMe test, the media guidance application may identify that the first user has certain genetic traits consistent with a certain culture, heritage, or race, such as genetic traits of an Ashkenazi Jew. Additionally, the media guidance application may identify a genetic disorder prevalent in Ashkenazi Jewish populations, such as Tay-Sachs. Further to this example, the media guidance application may generate a playlist for the first user that has content related to Ashkenazi Jews and additional content related to Tay-Sachs disease.

The media guidance application may access a list of users associated with the first user. For example, the media guidance application may store this list of users in local memory. Furthermore, the media guidance application may receive requests from various users of a local device and records and maintains a log of users who frequently interact with the system. For example, the media guidance application may access user profiles for a wife, daughter, and son, stored in the same location of memory as that for the first user, the father. Furthermore, the media guidance application may identify that the father is associated with his wife, daughter, and son, respectively.

The media guidance application may compare genetic data of each user in the list of users with genetic data of the first user. For example, the media guidance application may request access to and retrieve genetic data from 23andMe for users linked to the first user, the father. Furthermore, the media guidance application may retrieve 23andMe results and analysis that compare the genetic data of the father with his wife, daughter, and son, respectively, to identify a genetic link to the father's DNA.

The media guidance application may determine, based on the comparing, a subset of the list of users that has a genetic link to the first user. For example, the media guidance application may determine from the comparison of the father's genetic data to the genetic data of the wife, daughter, and son, that the daughter and son have a genetic link to the father.

The media guidance application may determine for each given user in the subset a likelihood that the given user in the subset has a genetic link to the disease based on the genetic data associated with the given user in the subset. For example, the media guidance application may independently compare the genetic data, retrieved from 23andMe, of the father with the genetic data of the daughter and son, respectively. Furthermore, the media guidance application may determine, based off the comparison found in the 23andMe results and analysis, the daughter has a 50% likelihood of having a genetic link to Alzheimer's disease. Additionally, the media guidance application may determine, based on the comparison found in the 23andMe results and analysis, the son has a 60% likelihood of having a genetic link to Alzheimer's disease.

The media guidance application may, in response to determining the likelihood of each user in the subset, select a second user within the subset that is associated with a likelihood that exceeds a threshold. For example, the media guidance application may determine a threshold likelihood value of 55%. Furthermore, the media guidance application may compare the likelihood values of the daughter and son, who are genetically linked to the first user, the father, with the threshold value. The media guidance application may determine, the son's likelihood value of having a genetic link to Alzheimer's disease exceeds the threshold value. Subsequently, the media guidance application may select the son as a second user.

The media guidance application may recommend disease-related content to the selected second user that is determined to have a genetic link to the first user and that is associated with a likelihood of having a genetic link to the disease that exceeds the threshold. For example, the media guidance application may recommend Alzheimer's disease-related content to the second user, the son. Furthermore, the media guidance application may recommend content such as documentaries, shows or movies related to Alzheimer's disease. In some embodiments, the media guidance application may determine a first time when the first user became aware of having genomic sequencing data that is associated with the disease. For example, the media guidance application may create a first time stamp of the time and date of when the first user, the father, became aware of his genomic data sequencing results. In some embodiments, the media guidance application may compare the first time to a current time to determine a length of time since the first user became aware of having genomic sequencing data that is associated with the disease. For example, the media guidance application may compare the first time stamp with the current time stamp and determine the difference to be two weeks. Furthermore, in some embodiments, the media guidance application may, in response to determining that the length of time exceeds a threshold, generate for display the recommended disease-related content. For example, the media guidance application may set a threshold time value of one week, meaning that if the interval in which the first user, the father, does not view Alzheimer's disease-related content exceeds one week, the media guidance application may generate for display Alzheimer's related content for the father.

In some embodiments, the media guidance application may search a social network for user-generated content associated with the disease. For example, based on the father's high likelihood of having a genetic-link to Alzheimer's disease, the media guidance application may search social media platforms, such as YouTube, Facebook, Instagram, etc., for user-generated content related to Alzheimer's disease. In some additional embodiments, the media guidance application may search for non-friends, meaning people with whom the user does not have an official connection, who have similar disease-related situations. For example, the media guidance application may search social networks such as Facebook or Twitter to find individuals posting about father-son relationships coping with Alzheimer's disease. Additionally, the media guidance application may identify and recommend chat rooms that are designated for fathers and sons who are dealing with Alzheimer's disease in some way.

In some embodiments, the media guidance application may cause the user generated content to be presented to the first and second users as the recommendation. For example, the media guidance application may retrieve relevant user generated Alzheimer's disease-related content. An example of relevant user generated content may be posts or short video clips where people give firsthand accounts about the struggles of living with Alzheimer's disease. Additionally, the media guidance application may present the retrieved Alzheimer's related content to the first user, the father, and the second user, the son.

In some embodiments, the media guidance application may determine an age of an offspring of the first user. For example, as previously described, the first user, the father, may have a high likelihood for developing Alzheimer's disease. The father may also have a genetic link to a second user, his son. Furthermore, the media guidance application may identify the age of the son.

In some embodiments, the media guidance application may retrieve genetic testing data of the first user. For example, the media guidance application may retrieve the father's genetic testing data. In some embodiments, the media guidance application may generate for display, a list of diseases from the first user's genetic test results. For example, the media guidance application may identify various diseases from the father's genetic testing results, one of which is Alzheimer's disease. Furthermore, in some embodiments, the media guidance application may request the first user to evaluate which diseases, from the genetic testing results, are age-appropriate for the offspring. For example, the media guidance application may identify the age of the son to be seven years old. The media guidance application may then request that the father evaluate the recommended disease-related content to ensure it is age-appropriate for the son. The media guidance application may receive a request from the father to not generate for display any content related to pictures or video of surgeries depicting blood or otherwise gruesome content. In some embodiments, the media guidance application may, based off the first user's evaluation of appropriate content based on offspring age, prevent disease-related content from being generated for display that is inappropriate for the offspring. For example, the media guidance application, based off the father's request to prevent content related to open surgery, may not generate for display a documentary that follows an experimental brain surgeon's attempt at deep brain stimulation of an Alzheimer's patient.

In some embodiments, the media guidance application may determine whether an additional user is within a perceptible range of the recommended disease-related content. For example, the media guidance application may utilize facial recognition technology to identify an additional user in view of a television.

In some embodiments, the media guidance application may determine an identity of the additional user. For example, the media guidance application may determine the additional user is a friend of the previously described son. In some embodiments, the media guidance application may retrieve genetic data of the additional user. The media guidance application may retrieve the genetic data of the additional user, the friend. Furthermore, in some embodiments, the media guidance application may determine, based on the genetic data, whether the additional user has a genetic link to the first user. For example, the media guidance application may determine the friend of the son has no genetic link to the father. The media guidance application may recognize a need to block private disease-related information, preventing the friend from accessing sensitive family health information. For example, the media guidance application may block any recommended Alzheimer's-related content, since the private health information is not appropriate to share with the friend.

In some embodiments, the media guidance application may create an avatar in the likeness of the first user. For example, the media guidance application may use three-dimensional (3D) scanning functionality to create an avatar in the likeness of the first user, the father. Furthermore, the media guidance application may create an avatar that can communicate with viewers in a perceptible range of a television or similar viewing apparatus. In some embodiments, the media guidance application may present the disease-related content to the second user using the avatar. For example, the media guidance application may, in an effort to make disease-related content more relatable to the second user, the son, present the Alzheimer's disease-related content to the son using the father's avatar.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1 shows an illustrative example of an informational display for disease-related content generated for display by a media guidance application, in accordance with some embodiments of the disclosure;

FIGS. 2 and 3 show illustrative examples of display screens generated by a media guidance application in accordance with some embodiments of the disclosure;

FIG. 4 is a block diagram of an illustrative user equipment device in accordance with some embodiments of the disclosure;

FIG. 5 is a block diagram of an illustrative media system in accordance with some embodiments of the disclosure;

FIG. 6 shows a flowchart of an illustrative process for recommending disease-related content to users in accordance with some embodiments of the disclosure;

FIG. 7 shows an illustrative example for identifying disease traits of users and presenting disease-related content in accordance with some embodiments of the disclosure;

FIG. 8 shows a flowchart for generating a playlist of content based on a certain disease in accordance with some embodiments of the disclosure;

FIG. 9 shows a flowchart of an illustrative process for blocking disease related-content to underage or non-genetically linked users in accordance with some embodiments of the disclosure;

FIG. 10 shows a flowchart of an illustrative process for preventing age-inappropriate disease-related content in accordance with some embodiments of the disclosure;

FIG. 11 shows a flowchart of an illustrative process for recommending disease related content to the user in accordance with some embodiments of the disclosure;

FIG. 12 shows a flowchart of an illustrative process for accessing genomic sequencing databases in accordance with some embodiments of the disclosure; and

FIG. 13 shows a flowchart of an illustrative process for determining disease likelihood and threshold metrics in accordance with some embodiments of the disclosure.

### DETAILED DESCRIPTION

Accordingly, systems and methods are described herein for a media guidance application that recommends content, featuring a genetically-identifiable disease, based on a positive determination of a genetic marker that suggests a likelihood of having a certain disease. For example, if a first user who has offspring, such as a father in a family with two children, has genetic variations that indicate a high likelihood of developing Alzheimer's disease, which is a neurological disease that develops, later in life, the systems and methods provided herein selectively recommend content to users with a genetic-link to the father, such as the father's son, about Alzheimer's disease. Thus, the father or any first user, for that matter, can have assistance in communicating sensitive health topics with family members about the risks and realities of certain genetic diseases.

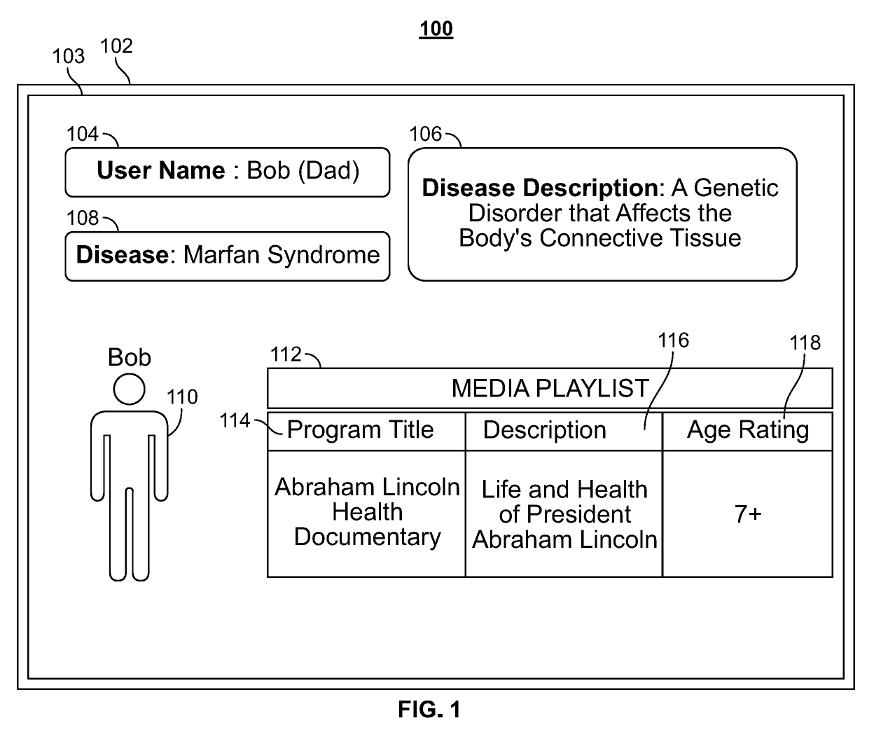

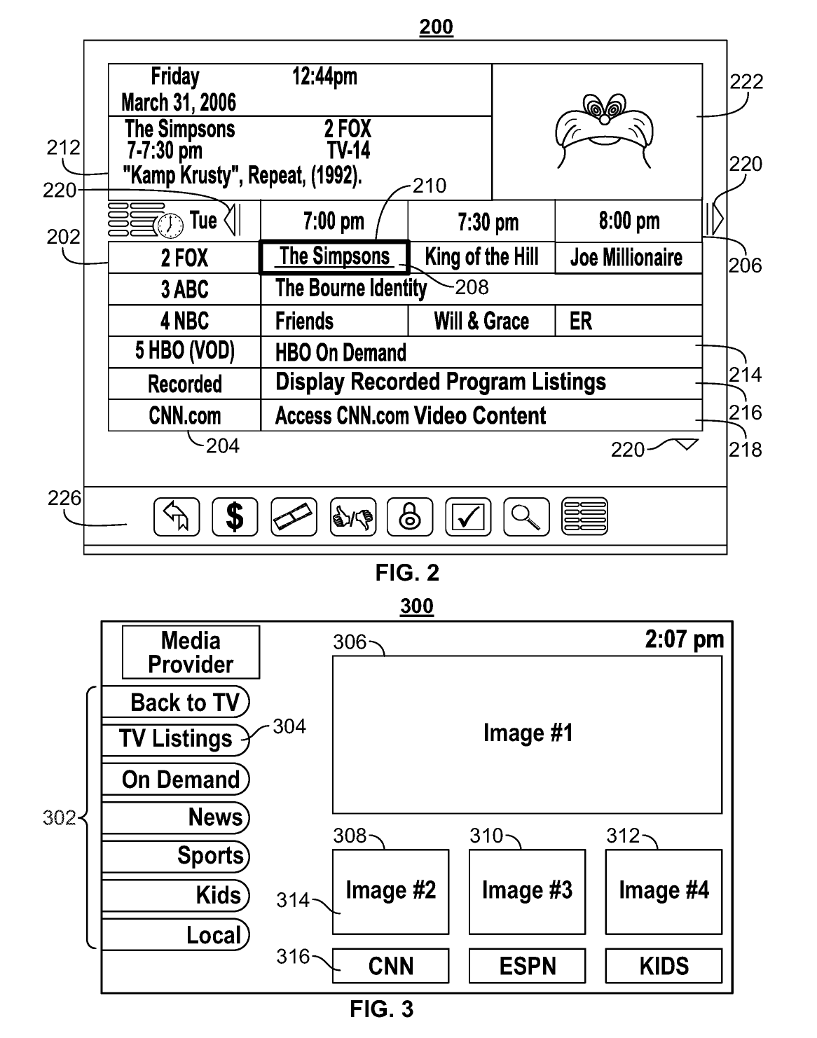

FIG. 1 shows an illustrative example of an informational display of disease-related content generated for display by a media guidance application, in accordance with some embodiments of the disclosure. FIG. 1 shows illustrative user equipment 102, which operates control circuitry that executes a media guidance application. Details relating to user equipment 102, control circuitry, and the media guidance application are described in further detail below with respect to FIGS. 2-5.

As depicted in FIG. 1, the media guidance application generates for display disease display 103 (e.g., to be displayed by a display of user equipment 102). Disease display 103 depicts the genetic disease of a user. As used herein, a “genetic disease” (also referred to interchangeably herein as a “disease”) is defined as an inherited medical condition caused by a DNA abnormality, that can be identified through genetic testing. As used herein, a “genetic test” is defined as a test that allows the determination of bloodlines and the genetic diagnosis of vulnerabilities to inherited diseases (e.g., 23andMe testing kits, Ancestry.com testing kits, etc.). For example, genetic diseases can be a number of diseases, such as Down Syndrome, Cystic fibrosis, Huntington's disease, Sickle cell anemia, Hemophilia, and certain types of cancer.

The media guidance application may include, within disease display 103, user name tile 104 that indicates the name of the user. For example, the name represented in name tile 104 may be a person's given name, nickname, or family unit designation (e.g., dad, mom, son, etc.). The user designated in user name tile 104 may also be genetically-linked to other users who may view disease display 103. As used herein, “genetically-linked” is defined as belonging to the same bloodline and possessing inherited genetic traits. Additionally, the remaining content located in disease display 103 is related to the user designated in user name tile 104. The media guidance application may also generate for display within or adjacent to disease display 103, disease description tile 106, which gives a brief explanation of the disease of the user designated in user name tile 104. The media guidance application may also generate for display within or adjacent to disease display 103 disease identifier tile 108, which represents a disease which the user, designated in user name tile 104, has a high likelihood of developing.

The media guidance application may generate for display, within disease display 103, avatar 110, which communicates disease-related information to genetically-linked users. As used herein, “avatar” is a term that may designate a visual representation of a user created in that user's likeness, which can communicate with other users viewing disease display 103.

The media guidance application may generate for display, within disease display 103, media playlist 112, which presents disease-related content. The term “disease-related content” in relation to “media playlist 112,” and as otherwise used in this disclosure, is used merely for convenience, and refers to any form of time-shifted content, including but not limited to downloaded content, content accessible by way of a remote server, on-demand content, over-the-top content (described further below with respect to FIGS. 2-5), or any other type of time-shifted media asset. Furthermore, as used herein, “playlist” refers to a list of selectable media assets that may be generated for display and played one after another in a defined order automatically. The media guidance application may include within media playlist 112 any media asset relating to the disease of the user designated in user name tile 104 caused to be time-shifted, or caused to be added to a directory associated with the user. Media playlist 112 may include any form of media asset, including documentaries, blogs, shows, movies, videos, etc. The media guidance application may time-shift media assets, or may otherwise cause media assets to be indicated in media playlist 112, based on information determined from a user profile associated with the user, such as information derived from a 23andMe genetic test, where 23andMe is a company that facilitates at-home genetic testing.

As depicted in FIG. 1, media playlist 112 optionally includes program title column 114, which designates the titles of the programs related to the disease, and program description column 116, which gives a brief description of the associated program with disease-related content to give the user a better understanding of the program listing. Media playlist 112 may also include age column 118, which may designate the age range appropriate for viewing the disease-related content.

In some embodiments, a media guidance application (e.g., executed by control circuitry of user equipment or of a server remote from user equipment) may request access to a first user's genomic sequencing testing data, which is stored in an entity's database (e.g., 23andMe, Ancestry.com, Living DNA, etc.). As used herein, “first user” is defined as a user whose genetic testing data will be used for subsequent comparison and be the basis for presenting information and recommending content as depicted in disease display 103. Functionality of control circuitry, user equipment, servers, and communications networks is described in further detail below with respect to FIGS. 2-5. For example, the media guidance application may request a user's genetic testing data for a variety of reasons, including user input at a television, user input from an application on a wireless device, preset settings on a local device set to request genetic testing data at predetermined intervals, etc. Additionally, for example, the media guidance application may identify the first user in a variety of ways including user input via remote device or application designating first user status, facial recognition software that identifies users and cross-references those users with stored images of potential first users, or voice recognition with similar comparative techniques as described for facial recognition. In an additional example, the media guidance application may store a profile in a database stored locally and/or remotely on a service provider server that includes information for the users on a family service plan. Furthermore, the media guidance application may receive a request from a father in the aforementioned family to retrieve his 23andMe genetic testing data. As referred to herein, “genetic testing data,” is defined as the results of rigorous analysis of an individual's genome, i.e., the genetic material of an organism, including, among other things, likelihoods of developing certain diseases. Additionally, companies such as 23andMe produce results and analysis for each genetic test that can be accessed remotely in 23andMe's database. Furthermore, 23andMe's database of genetic testing data is produced through careful analysis and statistical methods. The data found in 23andMe's database is robust and simply need be extracted in order to obtain and discern possible outcomes for individual users and those user's genetically-linked offspring.

The media guidance application may, in response to requesting the first user's genomic sequencing testing data, retrieve from a profile associated with the first user the first user's genomic sequencing testing data. For example, as previously introduced, the media guidance application may access user profiles stored locally and/or remotely, one of which is associated with a first user, the father. In a further example, the media guidance application may request the father's 23andMe genomic sequencing test results and analysis from 23andMe's database. For example, the media guidance application may request access to the 23andMe database. Furthermore, the media guidance application may use a personal identifier, unique to the father, that is stored in the user profile, to request the father's genetic data, once the database grants access. The media guidance application may then access the father's 23andMe results and analysis and use the results and analysis to identify variations in the father's chromosomes, genes, and proteins, that have already been processed by the genetic testing entity. Furthermore, the media guidance application may identify, from the genetic testing entity's data, the name of a disease, which may appear in disease tile 108 and a description of the disease, which may appear in disease description tile 106.

The media guidance application may search, based on the retrieved first user's genomic sequencing testing data, a database that associates genomic sequencing data with diseases to identify a disease associated with the first user. For example, the media guidance application may access and search a 23andMe database that associates the father's genomic testing results with various diseases, which may, at various times, appear in disease display 103, in disease tile 108 and disease description tile 106. Furthermore, the media guidance application may determine from the information gathered from the directory of diseases that the father has a genetic variation associated with Alzheimer's disease, a neurological disorder. In some embodiments, the media guidance application may identify a genetic-related disease, which is a disorder or condition caused by abnormalities in a person's genome. For example, the media guidance application may search a database of genomic information that has various diseases associated with abnormalities in an individual's genome. In some embodiments, the media guidance application may identify a genomic abnormality, wherein a genome further comprises genetic material of an organism, such as a human, that contains information about that organism's traits and characteristics, including the organism's likelihood of having a certain disease. For example, the media guidance application may retrieve the father's genetic data from the 23andMe company database. The media guidance application may then parse through the data by using a keyword search algorithm that identifies diseases in the data.

In some embodiments, the media guidance application may present results to the first user based on the analysis, which indicates at least one of a likelihood that the first user is a carrier of a particular genetic mutation; the first user is associated with an increased risk of developing a disease, such as cancer, in the future; and a need for further testing. For example, the media guidance application may present results to the first user in disease display 103, the father, indicating the likelihood that he is a carrier for a certain disease. The media guidance application may parse through local and remote databases of time-shifted media assets using a keyword algorithm that retrieves content related to a certain disease. The media guidance application may retrieve movies, shows, documentaries, and various other media. In some embodiments, the media guidance application may generate content for presentation to the first user based on the genetic data of the first user. For example, the media guidance application may search and retrieve, as previously described, various media content to identify Alzheimer's-related content for the father. Furthermore, the media guidance application may present the Alzheimer's related content to the father in disease display 103. Specifically, the media guidance application may present the father's name in user name tile 104; the disease, Alzheimer's Disease, in disease tile 108; and a description of Alzheimer's disease in disease description tile 106. In an additional example, the media guidance application may request access to a remote database of Alzheimer's-related content. After receiving access to the database, the media guidance application may retrieve Alzheimer's disease-related content based off the user's profile stored locally and/or remotely. The media guidance application may use a keyword search algorithm to find the most suitable programming. An example of such a keyword search may include keywords such as father, Alzheimer's, family, prevention, etc. Subsequently, the media guidance application may retrieve a media asset profile for the Alzheimer's disease-related program that contains information such as title, a description, ratings, etc. The media guidance application may then populate program title column 114 with titles of Alzheimer's-related media assets, such as a documentary called, “Do You Know What My Name Is?” Furthermore, the media guidance application may also populate program description column 116 with a description, such as, “Real-life drama of how a Cleveland, Ohio, nursing home implemented a program designed to improve the lives of dementia patients.” Additionally, the media guidance application may retrieve ratings information for the documentary indicating the documentary is suitable for ages ten and older. The media guidance application may populate age rating column 118 with “10+” to designate the suggested age appropriate range for the documentary.

In some embodiments, the media guidance application may generate a playlist, as seen in media playlist 112, of content that includes a first media asset corresponding to a background of the first user based on the genetic data and a second media asset corresponding to the disease. For example, based on the results and analysis of the 23andMe test retrieved from the 23andMe database, the media guidance application may identify the first user has certain genetic traits consistent with a certain culture, heritage, or race, such as genetic traits of an Ashkenazi Jew. Additionally, the media guidance application may identify, by accessing supplemental resources from the 23andMe database that provide general knowledge about various diseases, a genetic disorder prevalent in Ashkenazi Jewish populations, such as Tay-Sachs, which is a degenerative neurological disorder. Further to this example, the media guidance application may generate a playlist for the first user that has content related to Ashkenazi Jews and additional content related to Tay-Sachs disease, where program title column 114, program description column 116, and age rating column 118 are populated in a fashion similar to that previously described.

The media guidance application may access a list of users associated with the first user. For example, the media guidance application may store this list of users in local memory and/or remotely in memory, as previously described. The media guidance application may use location algorithms to determine which users may have associations with other users based on frequency of television usage in a given geographical area, i.e., if certain people frequently watch media on the same device in the same room, at various times. Furthermore, the media guidance application may receive requests from various users of a local device and records and maintains a log of users who frequently interact with the system. For example, the media guidance application may access user profiles for a wife, daughter, and son, stored in the same location of memory as the first user, the father. Furthermore, the media guidance application may identify that the father is associated with his wife, daughter, and son, respectively.

The media guidance application may compare genetic data of each user in the list of users with genetic data of the first user. For example, the media guidance application may request access to and retrieve genetic data from 23andMe for users linked to the first user, the father. In this example, using the same technique as previously described with the unique identifier, the media guidance application may access the genetic data of the mother, daughter, and son. Furthermore, the media guidance application may retrieve 23andMe results and analysis that compare the genetic data of the father with his wife, daughter, and son, respectively, to identify a genetic link to the father's DNA. For example, the media guidance application may retrieve supplemental data from 23andMe's database that curates the comparisons made amongst the various users into a single outcome that clearly depicts the results of all associated users.

The media guidance application may determine, based on the comparing, a subset of the list of users that has a genetic link to the first user. For example, the media guidance application may access the supplemental data and using a keyword search algorithm, may identify a subset of the users who are genetically-linked to the father. The media guidance application may then determine from the comparison of the father's genetic data to the genetic data of the wife, daughter, and son, that the daughter and son have a genetic link to the father.

The media guidance application may determine for each given user in the subset a likelihood that the given user in the subset have a genetic link to the disease based on the genetic data associated with the given user in the subset. For example, the media guidance application may access the supplemental curated data, retrieved from 23andMe, of the genetically-linked subset and identify within the data the likelihood a user in the subset may have a genetic link to the disease. Furthermore, the media guidance application may determine, based off the comparison found in the 23andMe results and analysis, the daughter has a 50% likelihood of having a genetic link to Alzheimer's disease. Additionally, the media guidance application may determine, based off the comparison found in the 23andMe results and analysis, the son has a 60% likelihood of having a genetic link to Alzheimer's disease.

The media guidance application may, in response to determining the likelihood of each user in the subset, select a second user within the subset that is associated with a likelihood that exceeds a threshold. For example, the media guidance application may determine a threshold likelihood value of 55%. Furthermore, the media guidance application may compare the likelihood values of the daughter and son, who are genetically linked to the first user, the father, with the threshold value. The media guidance application may determine the son's likelihood value of having a genetic link to Alzheimer's disease exceeds the threshold value. Subsequently, the media guidance application may select the son as a second user. In a similar scenario, the media guidance application may determine from the retrieved genetic testing data, a new set of likelihood for a different disease, such as skin cancer. The media guidance application may identify the likelihoods for developing skin cancer to be much smaller, say, 10% each for both the daughter and son. The media guidance application may also retrieve data from 23andMe stating that the skin cancer threshold is 50%. The media guidance application may then determine neither the daughter nor the son may be selected in this circumstance.

The media guidance application may recommend, as depicted in disease display 103, disease-related content to the selected second user that is determined to have a genetic link to the first user and that is associated with a likelihood of having a genetic link to the disease that exceeds the threshold. For example, the media guidance application may recommend Alzheimer's disease-related content to the second user, the son. Furthermore, the media guidance application may recommend content such as documentaries, shows or movies related to Alzheimer's disease. The media guidance application may retrieve the recommendations from local and remote servers as previously described when discussing retrieving time-shifted media assets.

In some embodiments, the media guidance application may determine a first time when the first user became aware of having genomic sequencing data that is associated with the disease. As used herein, “first time” is defined as the initial moment the first user is made aware of disease-specific results of the first user's genetic test. For example, the media guidance application may create a first time stamp, which may be stored in local or remote memory, of the time and date of when the first user, the father, became aware of his genomic data sequencing results. In some embodiments, the media guidance application may compare the first time to a current time to determine a length of time since the first user became aware of having genomic sequencing data that is associated with the disease. For example, the media guidance application may compare the first time stamp with the current time stamp and determine the difference to be two weeks. The media guidance application may determine the time difference by using a built-in calculator function. Furthermore, in some embodiments, the media guidance application may, in response to determining that the length of time exceeds a threshold, generate for display the recommended disease-related content. For example, the media guidance application may set a threshold time value of one week, which is intended to give the father a buffer of time to gather himself before learning more about Alzheimer's disease, meaning that if the interval in which the first user, the father, does not view Alzheimer's disease related content exceeds one week, the media guidance application may generate for display in disease display 103, Alzheimer's disease related content for the father.

In some embodiments, the media guidance application may search a social network for user generated content associated with the disease. As used herein, “user generated content” is defined as material published on social media platforms created by individuals. For example, based off the father's high likelihood of having a genetic link to Alzheimer's disease, the media guidance application may search social media platforms, such as YouTube, Facebook, Instagram, etc., for user generated content related to Alzheimer's disease. The media guidance application may, again, as previously described, use a keyword search algorithm that identifies Alzheimer's disease related content on various forms of social media. In some additional embodiments, the media guidance application may search for non-friends, meaning people with whom the user does not have an official connection, who have similar disease related situations. For example, the media guidance application may search social networks such as Facebook or Twitter to find individuals posting about father-son relationships coping with Alzheimer's disease. Additionally, the media guidance application may identify and recommend chat rooms that are designated for fathers and sons who are dealing with Alzheimer's disease in some way. The media guidance application may use these previously identified social media outlets to populate media playlist 112.

In some embodiments, the media guidance application may cause the user generated content to be presented to the first and second users as the recommendation. For example, the media guidance application may retrieve relevant user generated Alzheimer's disease related content. An example of relevant user generated content may be posts or short video clips where people give firsthand accounts about the struggles of living with Alzheimer's disease. Additionally, the media guidance application may present the retrieved Alzheimer's related content to the first user (e.g., the father) and the second user (e.g., the son). The media guidance application may generate for display disease display 103 which in turn will populate media playlist 112 with user generated social media content.

In some embodiments, the media guidance application may determine an age of an offspring of the first user. For example, as previously described, the first user (e.g., the father) may have a high likelihood for developing Alzheimer's disease. The father may also have a genetic link to a second user (e.g., the son). Furthermore, the media guidance application may identify the age of the son. The media guidance application may determine the age of the son by accessing a locally stored profile of the son, where he previously entered his age. The media guidance application may also determine the age of the user by searching the father's profile, using a camera that identifies facial signatures that identify a person, or facial recognition software that identifies users in a viewing region. The media guidance application may determine that the son is six years old.

In some embodiments, the media guidance application may retrieve genetic testing data of the first user. For example, the media guidance application may retrieve the father's genetic testing data as previously described. In some embodiments, the media guidance application may generate for display a list of diseases from the first user's (e.g., the father's) genetic test results. For example, the media guidance application may identify various diseases from the father's genetic testing results from the 23andMe database, one of which is Alzheimer's disease. Furthermore, in some embodiments, the media guidance application may request the first user to evaluate which diseases, from the genetic testing results, are age-appropriate for the offspring. For example, the media guidance application may identify the age of the son to be six years old. The media guidance application may then requests that the father evaluate the recommended disease related content to ensure it is age-appropriate for the son. Additionally, the media guidance application may provide suggested age ratings to give the father additional information about the content in question. The media guidance application may receive a request from the father to not generate for display any content related to pictures or video of surgeries depicting blood or otherwise gruesome content. In some embodiments, the media guidance application may, based off the first user's evaluation of appropriate content based on offspring age, prevent disease-related content from being generated for display that is inappropriate for the offspring. Additionally, the media guidance application may prevent age-inappropriate disease related content from being generated by retrieving the father's blocking preferences and marking those media assets as unwatchable for the son. However, the media guidance application may receive instruction from the father to allow the son to view more gruesome content on the son's tenth birthday. In this scenario, the media guidance application may create a setting that ceases to prevent previously restricted content from being generated for display to the son any time on or after his tenth birthday. The media guidance application may find the son's birthday information on the son's profile that is stored locally and/or remotely. For example, the media guidance application, based off the father's request to prevent content related to open surgery, may not generate for display a documentary that follows an experimental brain surgeon's attempt at deep brain stimulation of an Alzheimer's patient.

In some embodiments, the media guidance application may determine whether an additional user is within a perceptible range of the recommended disease-related content. For example, the media guidance application may utilize facial recognition technology to identify an additional user in view of a television. As previously mentioned, the media guidance application may use facial recognition technology to perform a comparison of users present within a viewing area of a television to make positive identifications.

In some embodiments, the media guidance application may determine an identity of the additional user. For example, the media guidance application may determine the additional user is a friend of the previously described son. Additionally, the media guidance application may determine the additional user is the daughter, who does have a genetic link. In some embodiments, the media guidance application may retrieve genetic data of the additional user. The media guidance application may retrieve the genetic data of the additional user, the friend, or the daughter in the separate example. Furthermore, in some embodiments, the media guidance application may determine, based on the genetic data, whether the additional user has a genetic link to the first user. For example, the media guidance application may determine the friend of the son has no genetic link to the father or that the daughter has a genetic link to the father. The media guidance application may recognize a need to block private disease-related information, preventing the friend from accessing sensitive family health information. For example, the media guidance application may block any recommended Alzheimer's related content, since the private health information is not appropriate to share with the friend. However, the media guidance application may display disease display 103 if the daughter, who is not genetically-linked to Alzheimer's disease but still genetically-linked to the father, is present.

In some embodiments, the media guidance application may create an avatar in the likeness of the first user. The media guidance application may have access to an avatar modeling software, where you upload a headshot of a user and it creates an avatar. For example, the media guidance application may use 3D scanning functionality to create an avatar in the likeness of the first user, the father. Furthermore, the media guidance application may create an avatar that can communicate with viewers in a perceptible range of a television or similar viewing apparatus. The media guidance application may enable the avatar to speak using previously inputted voice characteristics of the first user (e.g., the father). In some embodiments, the media guidance application may present the disease-related content to the second user using avatar 110. For example, the media guidance application may, in an effort to make disease-related content more relatable to the second user (e.g., the son) present the Alzheimer's disease related content to the son using the father's avatar. Additionally, the media guidance application may present the user the option to use the avatar for presentation may be optional and not. In a circumstance, the media guidance application may present the user with an option to use the avatar or not. Additionally, the media guidance application may give the user the option to either view content from media playlist 112 or from avatar 110. The media guidance application may also have a toggle setting that may allow or prevent the avatar from presenting.

The media guidance application and/or any instructions for performing any of the embodiments discussed herein may be encoded on computer readable media. Computer readable media includes any media capable of storing data. The computer readable media may be transitory, including, but not limited to, propagating electrical or electromagnetic signals, or may be non-transitory including, but not limited to, volatile and non-volatile computer memory or storage devices such as a hard disk, floppy disk, USB drive, DVD, CD, media cards, register memory, processor caches, Random Access Memory (“RAM”), etc.

With the advent of the Internet, mobile computing, and high-speed wireless networks, users are accessing media on user equipment devices on which they traditionally did not. As referred to herein, the phrase “user equipment device,” “user equipment,” “user device,” “electronic device,” “electronic equipment,” “media equipment device,” or “media device” should be understood to mean any device for accessing the content described above, such as a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a hand-held computer, a stationary telephone, a personal digital assistant (PDA), a mobile telephone, a portable video player, a portable music player, a portable gaming machine, a smart phone, or any other television equipment, computing equipment, or wireless device, and/or combination of the same. In some embodiments, the user equipment device may have a front facing screen and a rear facing screen, multiple front screens, or multiple angled screens. In some embodiments, the user equipment device may have a front facing camera and/or a rear facing camera. On these user equipment devices, users may be able to navigate among and locate the same content available through a television. Consequently, media guidance may be available on these devices, as well. The guidance provided may be for content available only through a television, for content available only through one or more of other types of user equipment devices, or for content available both through a television and one or more of the other types of user equipment devices. The media guidance applications may be provided as on-line applications (i.e., provided on a web-site), or as stand-alone applications or clients on user equipment devices. Various devices and platforms that may implement media guidance applications are described in more detail below.

One of the functions of the media guidance application is to provide media guidance data to users. As referred to herein, the phrase “media guidance data” or “guidance data” should be understood to mean any data related to content or data used in operating the guidance application. For example, the guidance data may include program information, guidance application settings, user preferences, user profile information, media listings, media-related information (e.g., broadcast times, broadcast channels, titles, descriptions, ratings information (e.g., parental control ratings, critic's ratings, etc.), genre or category information, actor information, logo data for broadcasters' or providers' logos, etc.), media format (e.g., standard definition, high definition, 3D, etc.), advertisement information (e.g., text, images, media clips, etc.), on-demand information, blogs, websites, and any other type of guidance data that is helpful for a user to navigate among and locate desired content selections.

FIGS. 2-3 show illustrative display screens that may be used to provide media guidance data. The display screens shown in FIGS. 2-3 may be implemented on any suitable user equipment device or platform. While the displays of FIGS. 2-3 are illustrated as full screen displays, they may also be fully or partially overlaid over content being displayed. A user may indicate a desire to access content information by selecting a selectable option provided in a display screen (e.g., a menu option, a listings option, an icon, a hyperlink, etc.) or pressing a dedicated button (e.g., a GUIDE button) on a remote control or other user input interface or device. In response to the user's indication, the media guidance application may provide a display screen with media guidance data organized in one of several ways, such as by time and channel in a grid, by time, by channel, by source, by content type, by category (e.g., movies, sports, news, children, or other categories of programming), or other predefined, user-defined, or other organization criteria.

FIG. 2 shows illustrative grid of a program listings display 200 arranged by time and channel that also enables access to different types of content in a single display. Display 200 may include grid 202 with: (1) a column of channel/content type identifiers 204, where each channel/content type identifier (which is a cell in the column) identifies a different channel or content type available; and (2) a row of time identifiers 206, where each time identifier (which is a cell in the row) identifies a time block of programming. Grid 202 also includes cells of program listings, such as program listing 208, where each listing provides the title of the program provided on the listing's associated channel and time. With a user input device, a user can select program listings by moving highlight region 210. Information relating to the program listing selected by highlight region 210 may be provided in program information region 212. Region 212 may include, for example, the program title, the program description, the time the program is provided (if applicable), the channel the program is on (if applicable), the program's rating, and other desired information.

In addition to providing access to linear programming (e.g., content that is scheduled to be transmitted to a plurality of user equipment devices at a predetermined time and is provided according to a schedule), the media guidance application also provides access to non-linear programming (e.g., content accessible to a user equipment device at any time and is not provided according to a schedule). Non-linear programming may include content from different content sources including on-demand content (e.g., VOD), Internet content (e.g., streaming media, downloadable media, etc.), locally stored content (e.g., content stored on any user equipment device described above or other storage device), or other time-independent content. On-demand content may include movies or any other content provided by a particular content provider (e.g., HBO On Demand providing “The Sopranos” and “Curb Your Enthusiasm”). HBO ON DEMAND is a service mark owned by Time Warner Company L.P. et al. and THE SOPRANOS and CURB YOUR ENTHUSIASM are trademarks owned by the Home Box Office, Inc. Internet content may include web events, such as a chat session or Webcast, or content available on-demand as streaming content or downloadable content through an Internet web site or other Internet access (e.g. FTP).

Grid 202 may provide media guidance data for non-linear programming including on-demand listing 214, recorded content listing 216, and Internet content listing 218. A display combining media guidance data for content from different types of content sources is sometimes referred to as a “mixed-media” display. Various permutations of the types of media guidance data that may be displayed that are different than display 200 may be based on user selection or guidance application definition (e.g., a display of only recorded and broadcast listings, only on-demand and broadcast listings, etc.). As illustrated, listings 214, 216, and 218 are shown as spanning the entire time block displayed in grid 202 to indicate that selection of these listings may provide access to a display dedicated to on-demand listings, recorded listings, or Internet listings, respectively. In some embodiments, listings for these content types may be included directly in grid 202. Additional media guidance data may be displayed in response to the user selecting one of the navigational icons 220. (Pressing an arrow key on a user input device may affect the display in a similar manner as selecting navigational icons 220.)

Display 200 may also include video region 222, and options region 226. Video region 222 may allow the user to view and/or preview programs that are currently available, will be available, or were available to the user. The content of video region 222 may correspond to, or be independent from, one of the listings displayed in grid 202. In some embodiments, display 200 may include a selectable option (not shown) to access a display of a list of scheduled recordings. For example, the media guidance application may generate for display the list of scheduled recordings that is similar to a display generated for display in response to a selection of third selectable option 128 of FIG. 1. In some embodiments, the display 110 of FIG. 1 may be generated for display over grid display 200 upon invoking the display 200, instead of over a video of a media asset. Grid displays including a video region are sometimes referred to as picture-in-guide (PIG) displays. PIG displays and their functionalities are described in greater detail in Satterfield et al. U.S. Pat. No. 6,564,378, issued May 13, 2003 and Yuen et al. U.S. Pat. No. 6,239,794, issued May 29, 2001, which are hereby incorporated by reference herein in their entireties. PIG displays may be included in other media guidance application display screens of the embodiments described herein.

Options region 226 may allow the user to access different types of content, media guidance application displays, and/or media guidance application features. Options region 226 may be part of display 200 (and other display screens described herein), or may be invoked by a user by selecting an on-screen option or pressing a dedicated or assignable button on a user input device. The selectable options within options region 226 may concern features related to program listings in grid 202 or may include options available from a main menu display. Features related to program listings may include searching for other air times or ways of receiving a program, recording a program, enabling series recording of a program, setting program and/or channel as a favorite, purchasing a program, or other features. Options available from a main menu display may include search options, VOD options, parental control options, Internet options, cloud-based options, device synchronization options, second screen device options, options to access various types of media guidance data displays, options to subscribe to a premium service, options to edit a user's profile, options to access a browse overlay, or other options.

The media guidance application may be personalized based on a user's preferences. A personalized media guidance application allows a user to customize displays and features to create a personalized “experience” with the media guidance application. This personalized experience may be created by allowing a user to input these customizations and/or by the media guidance application monitoring user activity to determine various user preferences. Users may access their personalized guidance application by logging in or otherwise identifying themselves to the guidance application. Customization of the media guidance application may be made in accordance with a user profile. The customizations may include varying presentation schemes (e.g., color scheme of displays, font size of text, etc.), aspects of content listings displayed (e.g., only HDTV or only 3D programming, user-specified broadcast channels based on favorite channel selections, re-ordering the display of channels, recommended content, etc.), desired recording features (e.g., recording or series recordings for particular users, recording quality, etc.), parental control settings, customized presentation of Internet content (e.g., presentation of social media content, e-mail, electronically delivered articles, etc.) and other desired customizations.

The media guidance application may allow a user to provide user profile information or may automatically compile user profile information. The media guidance application may, for example, monitor the content the user accesses and/or other interactions the user may have with the guidance application. For example, the media guidance application may determine through monitoring content the user access, the preference hierarchy of teams that was discussed in reference to the selection of second selectable option 126 in FIG. 1. Additionally, the media guidance application may obtain all or part of other user profiles that are related to a particular user (e.g., from other web sites on the Internet the user accesses, such as www.allrovi.com, from other media guidance applications the user accesses, from other interactive applications the user accesses, from another user equipment device of the user, etc.), and/or obtain information about the user from other sources that the media guidance application may access. As a result, a user can be provided with a unified guidance application experience across the user's different user equipment devices. This type of user experience is described in greater detail below in connection with FIG. 5. Additional personalized media guidance application features are described in greater detail in Ellis et al., U.S. Patent Application Publication No. 2005/0251827, filed Jul. 11, 2005, Boyer et al., U.S. Pat. No. 7,165,098, issued Jan. 16, 2007, and Ellis et al., U.S. Patent Application Publication No. 2002/0174430, filed Feb. 21, 2002, which are hereby incorporated by reference herein in their entireties.

Another display arrangement for providing media guidance is shown in FIG. 3. Video mosaic display 300 includes selectable options 302 for content information organized based on content type, genre, and/or other organization criteria. In display 300, television listings option 304 is selected, thus providing listings 306, 308, 310, and 312 as broadcast program listings. In some embodiments, the display 110 of FIG. 1 may be generated for display over grid display 300 upon invoking the display 300, instead of over a video of a media asset. In display 300 the listings may provide graphical images including cover art, still images from the content, video clip previews, live video from the content, or other types of content that indicate to a user the content being described by the media guidance data in the listing. Each of the graphical listings may also be accompanied by text to provide further information about the content associated with the listing. For example, listing 308 may include more than one portion, including media portion 314 and text portion 316. Media portion 314 and/or text portion 316 may be selectable to view content in full-screen or to view information related to the content displayed in media portion 314 (e.g., to view listings for the channel that the video is displayed on).

The listings in display 300 are of different sizes (i.e., listing 306 is larger than listings 308, 310, and 312), but if desired, all the listings may be the same size. Listings may be of different sizes or graphically accentuated to indicate degrees of interest to the user or to emphasize certain content, as desired by the content provider or based on user preferences. Various systems and methods for graphically accentuating content listings are discussed in, for example, Yates, U.S. Patent Application Publication No. 2010/0153885, filed Nov. 12, 2009, which is hereby incorporated by reference herein in its entirety.

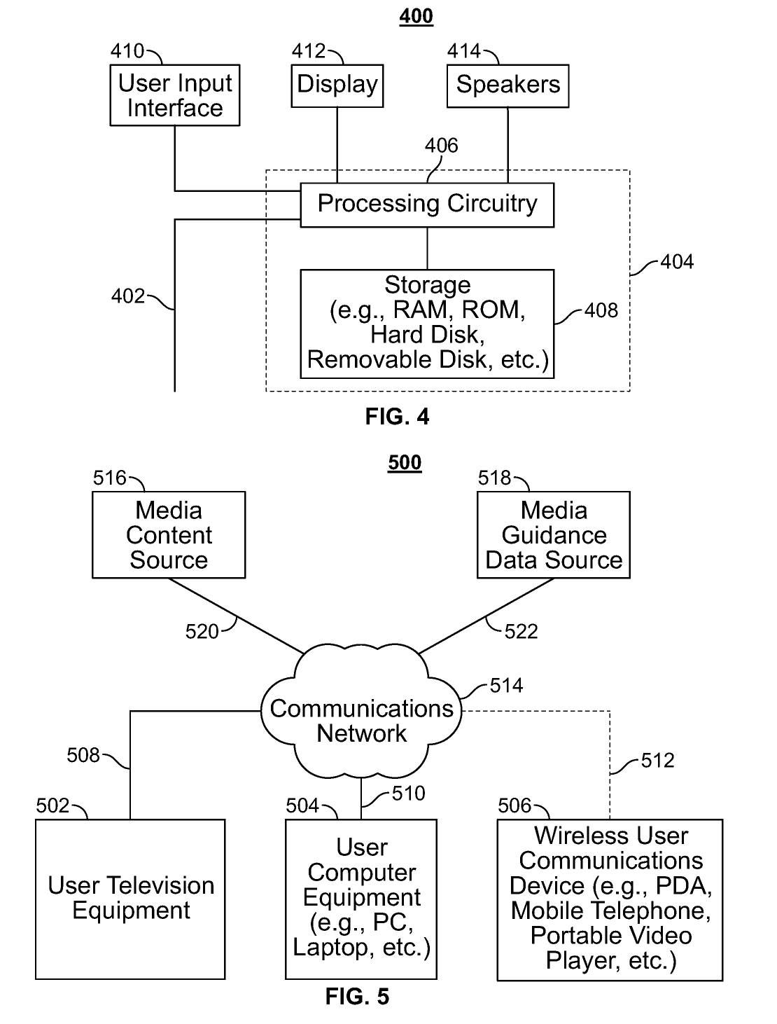

Users may access content and the media guidance application (and its display screens described above and below) from one or more of their user equipment devices. FIG. 4 shows a generalized embodiment of illustrative user equipment device 400. More specific implementations of user equipment devices are discussed below in connection with FIG. 5. User equipment device 400 may receive content and data via input/output (hereinafter “I/O”) path 402. I/O path 402 may provide content (e.g., broadcast programming, on-demand programming, Internet content, content available over a local area network (LAN) or wide area network (WAN), and/or other content) and data to control circuitry 404, which includes processing circuitry 406 and storage 408. Control circuitry 404 may be used to send and receive commands, requests, and other suitable data using I/O path 402. I/O path 402 may connect control circuitry 404 (and specifically processing circuitry 406) to one or more communications paths (described below). I/O functions may be provided by one or more of these communications paths, but are shown as a single path in FIG. 4 to avoid overcomplicating the drawing.

Control circuitry 404 may be based on any suitable processing circuitry such as processing circuitry 406. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, control circuitry 404 executes instructions for a media guidance application stored in memory (i.e., storage 408). Specifically, control circuitry 404 may be instructed by the media guidance application to perform the functions discussed above and below. For example, the media guidance application may provide instructions to control circuitry 404 to generate the media guidance displays. In some implementations, any action performed by control circuitry 404 may be based on instructions received from the media guidance application.

In client-server based embodiments, control circuitry 404 may include communications circuitry suitable for communicating with a guidance application server or other networks or servers. The instructions for carrying out the above mentioned functionality may be stored on the guidance application server. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths (which is described in more detail in connection with FIG. 5). In addition, communications circuitry may include circuitry that enables peer-to-peer communication of user equipment devices, or communication of user equipment devices in locations remote from each other (described in more detail below).

Memory may be an electronic storage device provided as storage 408 that is part of control circuitry 404. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage 408 may be used to store various types of content described herein as well as media guidance data described above. Nonvolatile memory may also be used (e.g., to launch a boot-up routine and other instructions). Cloud-based storage, described in relation to FIG. 5, may be used to supplement storage 408 or instead of storage 408.

Control circuitry 404 may include video generating circuitry and tuning circuitry, such as one or more analog tuners, one or more MPEG-2 decoders or other digital decoding circuitry, high-definition tuners, or any other suitable tuning or video circuits or combinations of such circuits. Encoding circuitry (e.g., for converting over-the-air, analog, or digital signals to MPEG signals for storage) may also be provided. Control circuitry 404 may also include scaler circuitry for upconverting and downconverting content into the preferred output format of the user equipment 400. Circuitry 404 may also include digital-to-analog converter circuitry and analog-to-digital converter circuitry for converting between digital and analog signals. The tuning and encoding circuitry may be used by the user equipment device to receive and to display, to play, or to record content. The tuning and encoding circuitry may also be used to receive guidance data. The circuitry described herein, including for example, the tuning, video generating, encoding, decoding, encrypting, decrypting, scaler, and analog/digital circuitry, may be implemented using software running on one or more general purpose or specialized processors. Multiple tuners may be provided to handle simultaneous tuning functions (e.g., watch and record functions, picture-in-picture (PIP) functions, multiple-tuner recording, etc.). If storage 408 is provided as a separate device from user equipment 400, the tuning and encoding circuitry (including multiple tuners) may be associated with storage 408.

A user may send instructions to control circuitry 404 using user input interface 410. User input interface 410 may be any suitable user interface, such as a remote control, mouse, trackball, keypad, keyboard, touch screen, touchpad, stylus input, joystick, voice recognition interface, or other user input interfaces. Display 412 may be provided as a stand-alone device or integrated with other elements of user equipment device 400. For example, display 412 may be a touchscreen or touch-sensitive display. In such circumstances, user input interface 410 may be integrated with or combined with display 412. Display 412 may be one or more of a monitor, a television, a liquid crystal display (LCD) for a mobile device, amorphous silicon display, low temperature poly silicon display, electronic ink display, electrophoretic display, active matrix display, electro-wetting display, electrofluidic display, cathode ray tube display, light-emitting diode display, electroluminescent display, plasma display panel, high-performance addressing display, thin-film transistor display, organic light-emitting diode display, surface-conduction electron-emitter display (SED), laser television, carbon nanotubes, quantum dot display, interferometric modulator display, or any other suitable equipment for displaying visual images. In some embodiments, display 412 may be HDTV-capable. In some embodiments, display 412 may be a 3D display, and the interactive media guidance application and any suitable content may be displayed in 3D. A video card or graphics card may generate the output to the display 412. The video card may offer various functions such as accelerated rendering of 3D scenes and 2D graphics, MPEG-2/MPEG-4 decoding, TV output, or the ability to connect multiple monitors. The video card may be any processing circuitry described above in relation to control circuitry 404. The video card may be integrated with the control circuitry 404. Speakers 414 may be provided as integrated with other elements of user equipment device 400 or may be stand-alone units. The audio component of videos and other content displayed on display 412 may be played through speakers 414. In some embodiments, the audio may be distributed to a receiver (not shown), which processes and outputs the audio via speakers 414.

The guidance application may be implemented using any suitable architecture. For example, it may be a stand-alone application wholly-implemented on user equipment device 400. In such an approach, instructions of the application are stored locally (e.g., in storage 408), and data for use by the application is downloaded on a periodic basis (e.g., from an out-of-band feed, from an Internet resource, or using another suitable approach). Control circuitry 404 may retrieve instructions of the application from storage 408 and process the instructions to generate any of the displays discussed herein. Based on the processed instructions, control circuitry 404 may determine what action to perform when input is received from input interface 410. For example, movement of a cursor on a display up/down may be indicated by the processed instructions when input interface 410 indicates that an up/down button was selected.

In some embodiments, the media guidance application is a client-server based application. Data for use by a thick or thin client implemented on user equipment device 400 is retrieved on-demand by issuing requests to a server remote to the user equipment device 400. In one example of a client-server based guidance application, control circuitry 404 runs a web browser that interprets web pages provided by a remote server. For example, the remote server may store the instructions for the application in a storage device. The remote server may process the stored instructions using circuitry (e.g., control circuitry 404) and generate the displays discussed above and below. The client device may receive the displays generated by the remote server and may display the content of the displays locally on equipment device 400. This way, the processing of the instructions is performed remotely by the server while the resulting displays are provided locally on equipment device 400. Equipment device 400 may receive inputs from the user via input interface 410 and transmit those inputs to the remote server for processing and generating the corresponding displays. For example, equipment device 400 may transmit a communication to the remote server indicating that an up/down button was selected via input interface 410. The remote server may process instructions in accordance with that input and generate a display of the application corresponding to the input (e.g., a display that moves a cursor up/down). The generated display is then transmitted to equipment device 400 for presentation to the user.

In some embodiments, the media guidance application is downloaded and interpreted or otherwise run by an interpreter or virtual machine (run by control circuitry 404). In some embodiments, the guidance application may be encoded in the ETV Binary Interchange Format (EBIF), received by control circuitry 404 as part of a suitable feed, and interpreted by a user agent running on control circuitry 404. For example, the guidance application may be an EBIF application. In some embodiments, the guidance application may be defined by a series of JAVA-based files that are received and run by a local virtual machine or other suitable middleware executed by control circuitry 404. In some of such embodiments (e.g., those employing MPEG-2 or other digital media encoding schemes), the guidance application may be, for example, encoded and transmitted in an MPEG-2 object carousel with the MPEG audio and video packets of a program.

User equipment device 400 of FIG. 4 can be implemented in system 500 of FIG. 5 as user television equipment 502, user computer equipment 504, wireless user communications device 506, or any other type of user equipment suitable for accessing content, such as a non-portable gaming machine. For simplicity, these devices may be referred to herein collectively as user equipment or user equipment devices, and may be substantially similar to user equipment devices described above. User equipment devices, on which a media guidance application may be implemented, may function as a standalone device or may be part of a network of devices. Various network configurations of devices may be implemented and are discussed in more detail below.

A user equipment device utilizing at least some of the system features described above in connection with FIG. 4 may not be classified solely as user television equipment 502, user computer equipment 504, or a wireless user communications device 506. For example, user television equipment 502 may, like some user computer equipment 504, be Internet-enabled allowing for access to Internet content, while user computer equipment 504 may, like some television equipment 502, include a tuner allowing for access to television programming. The media guidance application may have the same layout on various different types of user equipment or may be tailored to the display capabilities of the user equipment. For example, on user computer equipment 504, the guidance application may be provided as a web site accessed by a web browser. In another example, the guidance application may be scaled down for wireless user communications devices 506.

In system 500, there is typically more than one of each type of user equipment device but only one of each is shown in FIG. 5 to avoid overcomplicating the drawing. In addition, each user may utilize more than one type of user equipment device and also more than one of each type of user equipment device.

In some embodiments, a user equipment device (e.g., user television equipment 502, user computer equipment 504, wireless user communications device 506) may be referred to as a “second screen device.” For example, a second screen device may supplement content presented on a first user equipment device. The content presented on the second screen device may be any suitable content that supplements the content presented on the first device. In some embodiments, the second screen device provides an interface for adjusting settings and display preferences of the first device. In some embodiments, the second screen device is configured for interacting with other second screen devices or for interacting with a social network. The second screen device can be located in the same room as the first device, a different room from the first device but in the same house or building, or in a different building from the first device.

The user may also set various settings to maintain consistent media guidance application settings across in-home devices and remote devices. Settings include those described herein, as well as channel and program favorites, programming preferences that the guidance application utilizes to make programming recommendations, display preferences, and other desirable guidance settings. For example, if a user sets a channel as a favorite on, for example, the web site www.allrovi.com on their personal computer at their office, the same channel would appear as a favorite on the user's in-home devices (e.g., user television equipment and user computer equipment) as well as the user's mobile devices, if desired. Therefore, changes made on one user equipment device can change the guidance experience on another user equipment device, regardless of whether they are the same or a different type of user equipment device. In addition, the changes made may be based on settings input by a user, as well as user activity monitored by the guidance application.

The user equipment devices may be coupled to communications network 514. Namely, user television equipment 502, user computer equipment 504, and wireless user communications device 506 are coupled to communications network 514 via communications paths 508, 510, and 512, respectively. Communications network 514 may be one or more networks including the Internet, a mobile phone network, mobile voice or data network (e.g., a 4G or LTE network), cable network, public switched telephone network, or other types of communications network or combinations of communications networks. Paths 508, 510, and 512 may separately or together include one or more communications paths, such as, a satellite path, a fiber-optic path, a cable path, a path that supports Internet communications (e.g., IPTV), free-space connections (e.g., for broadcast or other wireless signals), or any other suitable wired or wireless communications path or combination of such paths. Path 512 is drawn with dotted lines to indicate that in the exemplary embodiment shown in FIG. 5 it is a wireless path and paths 508 and 510 are drawn as solid lines to indicate they are wired paths (although these paths may be wireless paths, if desired). Communications with the user equipment devices may be provided by one or more of these communications paths, but are shown as a single path in FIG. 5 to avoid overcomplicating the drawing.

Although communications paths are not drawn between user equipment devices, these devices may communicate directly with each other via communication paths, such as those described above in connection with paths 508, 510, and 512, as well as other short-range point-to-point communication paths, such as USB cables, IEEE 1394 cables, wireless paths (e.g., Bluetooth, infrared, IEEE 802-11x, etc.), or other short-range communication via wired or wireless paths. BLUETOOTH is a certification mark owned by Bluetooth SIG, INC. The user equipment devices may also communicate with each other directly through an indirect path via communications network 514.

System 500 includes content source 516 and media guidance data source 518 coupled to communications network 514 via communication paths 520 and 522, respectively. Paths 520 and 522 may include any of the communication paths described above in connection with paths 508, 510, and 512. Communications with the content source 516 and media guidance data source 518 may be exchanged over one or more communications paths, but are shown as a single path in FIG. 5 to avoid overcomplicating the drawing. In addition, there may be more than one of each of content source 516 and media guidance data source 518, but only one of each is shown in FIG. 5 to avoid overcomplicating the drawing. (The different types of each of these sources are discussed below.) If desired, content source 516 and media guidance data source 518 may be integrated as one source device. Although communications between sources 516 and 518 with user equipment devices 502, 504, and 506 are shown as through communications network 514, in some embodiments, sources 516 and 518 may communicate directly with user equipment devices 502, 504, and 506 via communication paths (not shown) such as those described above in connection with paths 508, 510, and 512.

Content source 516 may include one or more types of content distribution equipment including a television distribution facility, cable system headend, satellite distribution facility, programming sources (e.g., television broadcasters, such as NBC, ABC, HBO, etc.), intermediate distribution facilities and/or servers, Internet providers, on-demand media servers, and other content providers. NBC is a trademark owned by the National Broadcasting Company, Inc., ABC is a trademark owned by the American Broadcasting Company, Inc., and HBO is a trademark owned by the Home Box Office, Inc. Content source 516 may be the originator of content (e.g., a television broadcaster, a Webcast provider, etc.) or may not be the originator of content (e.g., an on-demand content provider, an Internet provider of content of broadcast programs for downloading, etc.). Content source 516 may include cable sources, satellite providers, on-demand providers, Internet providers, over-the-top content providers, or other providers of content. Content source 516 may also include a remote media server used to store different types of content (including video content selected by a user), in a location remote from any of the user equipment devices. Systems and methods for remote storage of content, and providing remotely stored content to user equipment are discussed in greater detail in connection with Ellis et al., U.S. Pat. No. 7,761,892, issued Jul. 20, 2010, which is hereby incorporated by reference herein in its entirety.

Media guidance data source 518 may provide media guidance data, such as the media guidance data described above. Media guidance data may be provided to the user equipment devices using any suitable approach. In some embodiments, the guidance application may be a stand-alone interactive television program guide that receives program guide data via a data feed (e.g., a continuous feed or trickle feed). Program schedule data and other guidance data may be provided to the user equipment on a television channel sideband, using an in-band digital signal, using an out-of-band digital signal, or by any other suitable data transmission technique. Program schedule data and other media guidance data may be provided to user equipment on multiple analog or digital television channels.

In some embodiments, guidance data from media guidance data source 518 may be provided to users' equipment using a client-server approach. For example, a user equipment device may pull media guidance data from a server, or a server may push media guidance data to a user equipment device. In some embodiments, a guidance application client residing on the user's equipment may initiate sessions with source 518 to obtain guidance data when needed, e.g., when the guidance data is out of date or when the user equipment device receives a request from the user to receive data. Media guidance may be provided to the user equipment with any suitable frequency (e.g., continuously, daily, a user-specified period of time, a system-specified period of time, in response to a request from user equipment, etc.). Media guidance data source 518 may provide user equipment devices 502, 504, and 506 the media guidance application itself or software updates for the media guidance application.

In some embodiments, the media guidance data may include viewer data. For example, the viewer data may include current and/or historical user activity information (e.g., what content the user typically watches, what times of day the user watches content, whether the user interacts with a social network, at what times the user interacts with a social network to post information, what types of content the user typically watches (e.g., pay TV or free TV), mood, brain activity information, etc.). The media guidance data may also include subscription data. For example, the subscription data may identify to which sources or services a given user subscribes and/or to which sources or services the given user has previously subscribed but later terminated access (e.g., whether the user subscribes to premium channels, whether the user has added a premium level of services, whether the user has increased Internet speed). In some embodiments, the viewer data and/or the subscription data may identify patterns of a given user for a period of more than one year. The media guidance data may include a model (e.g., a survivor model) used for generating a score that indicates a likelihood a given user will terminate access to a service/source. For example, the media guidance application may process the viewer data with the subscription data using the model to generate a value or score that indicates a likelihood of whether the given user will terminate access to a particular service or source. In particular, a higher score may indicate a higher level of confidence that the user will terminate access to a particular service or source. Based on the score, the media guidance application may generate promotions that entice the user to keep the particular service or source indicated by the score as one to which the user will likely terminate access.

Media guidance applications may be, for example, stand-alone applications implemented on user equipment devices. For example, the media guidance application may be implemented as software or a set of executable instructions which may be stored in storage 408, and executed by control circuitry 404 of a user equipment device 400. In some embodiments, media guidance applications may be client-server applications where only a client application resides on the user equipment device, and server application resides on a remote server. For example, media guidance applications may be implemented partially as a client application on control circuitry 404 of user equipment device 400 and partially on a remote server as a server application (e.g., media guidance data source 518) running on control circuitry of the remote server. When executed by control circuitry of the remote server (such as media guidance data source 518), the media guidance application may instruct the control circuitry to generate the guidance application displays and transmit the generated displays to the user equipment devices. The server application may instruct the control circuitry of the media guidance data source 518 to transmit data for storage on the user equipment. The client application may instruct control circuitry of the receiving user equipment to generate the guidance application displays.

Content and/or media guidance data delivered to user equipment devices 502, 504, and 506 may be over-the-top (OTT) content. OTT content delivery allows Internet-enabled user devices, including any user equipment device described above, to receive content that is transferred over the Internet, including any content described above, in addition to content received over cable or satellite connections. OTT content is delivered via an Internet connection provided by an Internet service provider (ISP), but a third party distributes the content. The ISP may not be responsible for the viewing abilities, copyrights, or redistribution of the content, and may only transfer IP packets provided by the OTT content provider. Examples of OTT content providers include YOUTUBE, NETFLIX, and HULU, which provide audio and video via IP packets. Youtube is a trademark owned by Google Inc., Netflix is a trademark owned by Netflix Inc., and Hulu is a trademark owned by Hulu, LLC. OTT content providers may additionally or alternatively provide media guidance data described above. In addition to content and/or media guidance data, providers of OTT content can distribute media guidance applications (e.g., web-based applications or cloud-based applications), or the content can be displayed by media guidance applications stored on the user equipment device. In some embodiments, the OTT sources may include Internet data sources such as RSS feeds, social media sources, news sources, or other sources that are updated at a more frequent interval than conventional media guidance data. For example, the OTT sources may include a database from which blobs of data (e.g., javascript object notation (JSON) data, or any other suitable data format) for sporting events may be queried and retrieved by user equipment devices 402, 404, and 406.

Media guidance system 500 is intended to illustrate a number of approaches, or network configurations, by which user equipment devices and sources of content and guidance data may communicate with each other for the purpose of accessing content and providing media guidance. The embodiments described herein may be applied in any one or a subset of these approaches, or in a system employing other approaches for delivering content and providing media guidance. The following four approaches provide specific illustrations of the generalized example of FIG. 5.

In one approach, user equipment devices may communicate with each other within a home network. User equipment devices can communicate with each other directly via short-range point-to-point communication schemes described above, via indirect paths through a hub or other similar device provided on a home network, or via communications network 514. Each of the multiple individuals in a single home may operate different user equipment devices on the home network. As a result, it may be desirable for various media guidance information or settings to be communicated between the different user equipment devices. For example, it may be desirable for users to maintain consistent media guidance application settings on different user equipment devices within a home network, as described in greater detail in Ellis et al., U.S. Patent Publication No. 2005/0251827, filed Jul. 11, 2005. Different types of user equipment devices in a home network may also communicate with each other to transmit content. For example, a user may transmit content from user computer equipment to a portable video player or portable music player.

In a second approach, users may have multiple types of user equipment by which they access content and obtain media guidance. For example, some users may have home networks that are accessed by in-home and mobile devices. Users may control in-home devices via a media guidance application implemented on a remote device. For example, users may access an online media guidance application on a website via a personal computer at their office, or a mobile device such as a PDA or web-enabled mobile telephone. The user may set various settings (e.g., recordings, reminders, or other settings) on the online guidance application to control the user's in-home equipment. The online guide may control the user's equipment directly, or by communicating with a media guidance application on the user's in-home equipment. Various systems and methods for user equipment devices communicating, where the user equipment devices are in locations remote from each other, is discussed in, for example, Ellis et al., U.S. Pat. No. 8,046,801, issued Oct. 25, 2011, which is hereby incorporated by reference herein in its entirety.

In a third approach, users of user equipment devices inside and outside a home can use their media guidance application to communicate directly with content source 516 to access content. Specifically, within a home, users of user television equipment 502 and user computer equipment 504 may access the media guidance application to navigate among and locate desirable content. Users may also access the media guidance application outside of the home using wireless user communications devices 506 to navigate among and locate desirable content.

In a fourth approach, user equipment devices may operate in a cloud computing environment to access cloud services. In a cloud computing environment, various types of computing services for content sharing, storage or distribution (e.g., video sharing sites or social networking sites) are provided by a collection of network-accessible computing and storage resources, referred to as “the cloud.” For example, the cloud can include a collection of server computing devices, which may be located centrally or at distributed locations, that provide cloud-based services to various types of users and devices connected via a network such as the Internet via communications network 514. These cloud resources may include one or more content sources 516 and one or more media guidance data sources 518. In addition or in the alternative, the remote computing sites may include other user equipment devices, such as user television equipment 502, user computer equipment 504, and wireless user communications device 506. For example, the other user equipment devices may provide access to a stored copy of a video or a streamed video. In such embodiments, user equipment devices may operate in a peer-to-peer manner without communicating with a central server.

The cloud provides access to services, such as content storage, content sharing, or social networking services, among other examples, as well as access to any content described above, for user equipment devices. Services can be provided in the cloud through cloud computing service providers, or through other providers of online services. For example, the cloud-based services can include a content storage service, a content sharing site, a social networking site, or other services via which user-sourced content is distributed for viewing by others on connected devices. These cloud-based services may allow a user equipment device to store content to the cloud and to receive content from the cloud rather than storing content locally and accessing locally-stored content.

A user may use various content capture devices, such as camcorders, digital cameras with video mode, audio recorders, mobile phones, and handheld computing devices, to record content. The user can upload content to a content storage service on the cloud either directly, for example, from user computer equipment 504 or wireless user communications device 506 having content capture feature. Alternatively, the user can first transfer the content to a user equipment device, such as user computer equipment 504. The user equipment device storing the content uploads the content to the cloud using a data transmission service on communications network 514. In some embodiments, the user equipment device itself is a cloud resource, and other user equipment devices can access the content directly from the user equipment device on which the user stored the content.

Cloud resources may be accessed by a user equipment device using, for example, a web browser, a media guidance application, a desktop application, a mobile application, and/or any combination of access applications of the same. The user equipment device may be a cloud client that relies on cloud computing for application delivery, or the user equipment device may have some functionality without access to cloud resources. For example, some applications running on the user equipment device may be cloud applications, i.e., applications delivered as a service over the Internet, while other applications may be stored and run on the user equipment device. In some embodiments, a user device may receive content from multiple cloud resources simultaneously. For example, a user device can stream audio from one cloud resource while downloading content from a second cloud resource. Or a user device can download content from multiple cloud resources for more efficient downloading. In some embodiments, user equipment devices can use cloud resources for processing operations such as the processing operations performed by processing circuitry described in relation to FIG. 4. In some embodiments, the cloud resources may include data sources such as RSS feeds, social media sources, news sources, or other sources that are updated at a more frequent interval than conventional media guidance data. For example, the cloud resources may provide data (e.g., JavaScript object notation (JSON) data, or any other suitable data format) for sporting events and may be queried and retrieved by user equipment devices 402, 404, and 406.

As referred herein, the term “in response to” refers to initiated as a result of. For example, a first action being performed in response to a second action may include interstitial steps between the first action and the second action. As referred herein, the term “directly in response to” refers to caused by. For example, a first action being performed directly in response to a second action may not include interstitial steps between the first action and the second action.

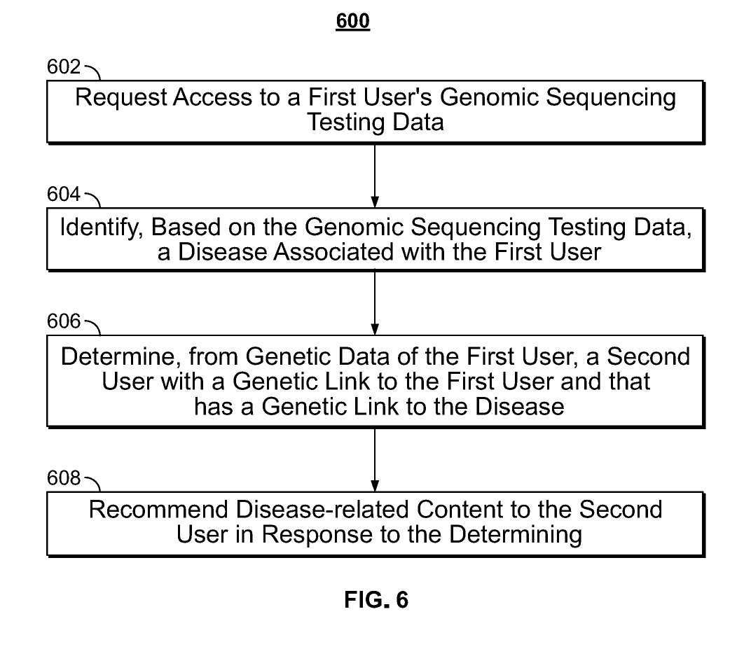

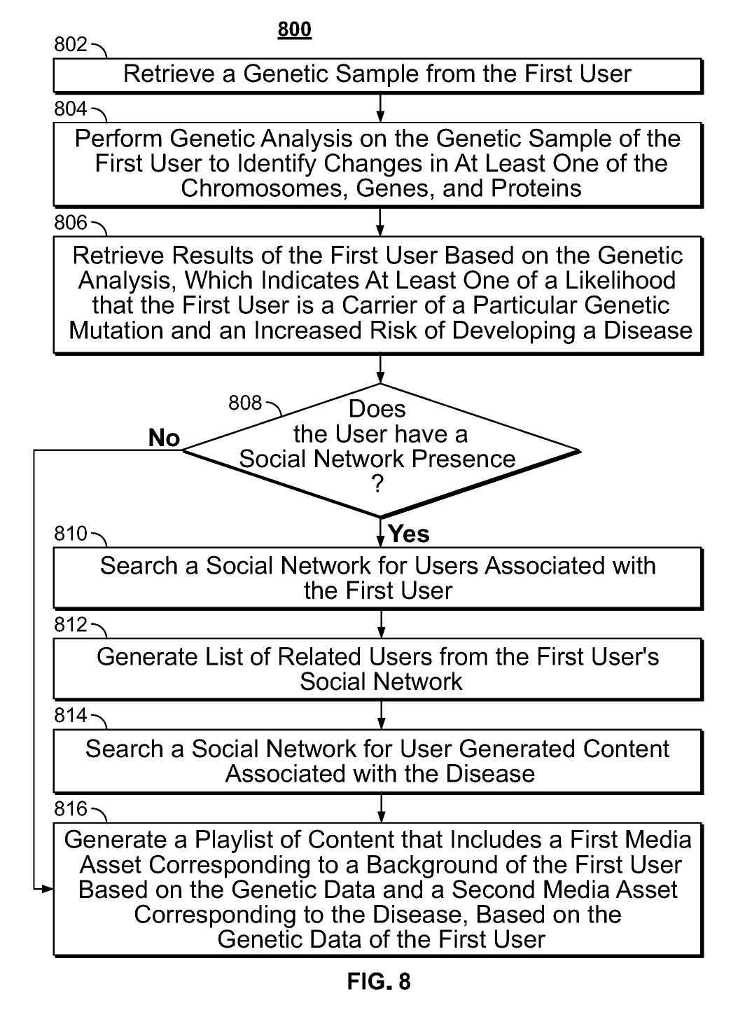

FIG. 6 is a flowchart of illustrative steps for recommending disease-related content to users in accordance with some embodiments of the disclosure. It should be noted that process 600 or any step thereof could be performed on, or provided by, any of the devices shown in FIGS. 4-5. For example, process 600 may be executed by control circuitry 404 (FIG. 4) as instructed by a media guidance application implemented on user equipment 102 (which may have the functionality of any or all of user equipment 502, 504, and/or 506 (FIG. 5)) in order to generate for display the results display. In addition, one or more steps of process 600 may be incorporated into or combined with one or more steps of any other process or embodiment (e.g., as described in relation to FIGS. 8-12).

At 602, the control circuitry 404 requests access to a first user's genomic sequencing testing data. For example, the media guidance application may request the previously mentioned father's 23andMe testing data, making the father the first user.

At 604, the control circuitry 404 identifies, based on the genomic sequencing testing data, a disease associated with the first user. For example, the media guidance application may identify that, based on the 23andMe genetic testing results, the father has a high likelihood of developing Alzheimer's disease.

At 606, the control circuitry 404 determines, from genetic data of the first user, a second user with a genetic link to the first user and that has a genetic link to the disease. For example, the media guidance application may retrieve supplemental data on other users that are genetically linked to the first user. The media guidance application may identify from the supplemental data that the father's son also has a high likelihood of developing Alzheimer's.

At 608, the control circuitry 404 recommends disease-related content to the second user in response to the determining. For example, the media guidance application may generate Alzheimer's disease related content for display to the son, after determining he also has a high likelihood of developing Alzheimer's disease.

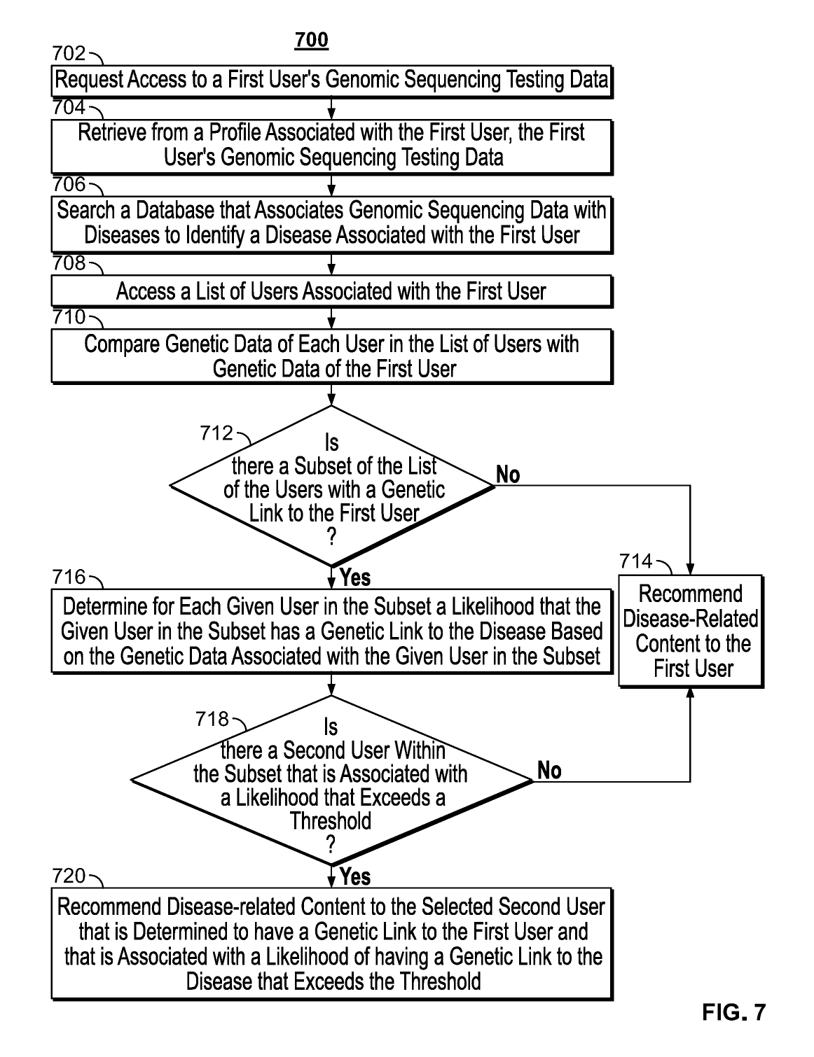

FIG. 7 is a flowchart of illustrative steps for identifying disease traits of users and presenting disease-related content in accordance with some embodiments of the disclosure. It should be noted that process 700 or any step thereof could be performed on, or provided by, any of the devices shown in FIGS. 4-5. For example, process 700 may be executed by control circuitry 404 (FIG. 4) as instructed by a media guidance application implemented on a user device (e.g., user equipment devices 502, 504, and/or 506 (FIG. 5)) in order to determine time-shifted media assets. In addition, one or more steps of process 700 may be incorporated into or combined with one or more steps of any other process or embodiment (e.g., as described in relation to FIGS. 8-13)).

At 702, the control circuitry 404 requests access to a first user's genomic sequencing testing data on communication network 514. For example, the media guidance application may request access to the first user's (e.g., the father's) 23andMe genetic testing data which contains results and analysis specific to his genome. The media guidance application may request access to the 23andMe database by connecting to the 23andMe network and submitting a user specific code that grants access to a person's genetic data. The media guidance application may then receive permission to access the father's genetic data.

At 704, the control circuitry 404 retrieves from a profile associated with the first user, the first user's genetic sequencing data. For example, the media guidance application may be granted access to the first user's (e.g., the father's) 23andMe profile from 23andMe's database of genetic information and data. The media guidance application may retrieve the genetic data by creating a link between the remote 23andMe database and the local storage apparatus, thus extracting the relevant genetic data from the source.

At 706, the control circuitry 404 searches a database that associates genomic sequencing data with diseases to identify a disease associated with the first user. For example, the media guidance application may identify, from the retrieved 23andMe testing data, that the father has a high likelihood of developing Alzheimer's disease. For example, the media guidance application may extract the 23andMe data which is arranged in a manner that highlights certain categories, one of such categories being specific to reporting disease likelihoods for a test subject. The media guidance application may search through that specific category and identify diseases highlighted within the data set.

At 708, the control circuitry 404 accesses a list of users associated with the first user. For example, the media guidance application may identify a family profile, as previously described. The media guidance application may identify within that profile users, such as the mom, daughter, and son, who are associated with the father.

At 710, the control circuitry 404 compares genetic data of each user in the list of users with genetic data of the first user. For example, the media guidance application may retrieve supplemental data from 23andMe that provides data on the associated users. For example, the media guidance application may retrieve data from 23andMe that contains information about associated users, such as lists of potential family members, offspring, relatives, etc. Furthermore, the media guidance application may retrieve further supplemental data, in a manner similar to that previously described, that contains comparative analyses on the various associated users and the father. The comparative analysis may contain results of statistical analyses that predict whether or not two individuals have a genetic link with each other and/or whether those who are genetically-linked have heightened risks of diseases.

At 712, the control circuitry 404 determines a subset of the list of the users with a genetic link to the first user. For example, the media guidance application may determine, from the 23andMe data, that the daughter and son have a genetic link to the father. The genetic testing data is curated and organized to explicitly outline various topics, once of which is a list of users who possess a genetic link and whether or not the additional user has a heightened risk of disease like the first user. Subsequently, the media guidance application may identify directly from the data set a list of linked individuals to the first user.

At 714, the control circuitry 404 recommends disease related content to the first user. For example, the media guidance application may not find an associated user with either a genetic link to the first user or a high enough likelihood of developing a disease, based on the 23andMe data previously mentioned and described. In this circumstance, the media guidance application may recommend disease-related content only to the first user. The media guidance application may determine the appropriate content for the first user by identifying the disease associated with the user, such as Alzheimer's disease. Subsequently, the media guidance application may perform a keyword searching algorithm, as previously described, that searches through local and/or remote databases of media assets, including social media outlets, cable service providers, or a variety of other subscription-based remote service providers. Once the media guidance application identifies the appropriate content to present to the first user, the media guidance application may populate media playlist 112, program title column 114, program description column 116 and age rating column 118.

At 716, the control circuitry 404 determines for each given user in the subset a likelihood that the given user in the subset has a genetic link to the disease based on the genetic data associated with the given user in the subset. For example, the media guidance application may determine, based on the 23andMe analysis and results, which, as previously described, specifically outlines related users and the users' individual likelihoods of having a high risk for the same diseases as the first user, that the daughter has a 10% likelihood of carrying the Alzheimer's mutation and the son has a 70% chance of having the Alzheimer's mutation.

At 718, the control circuitry 404 determines if a second user within the subset that is associated with a likelihood exceeds a threshold. For example, the media guidance application may determine, based off the 23andMe data and results, an acceptable threshold value to determine the severity of an individual's potential for developing a disease later in life. As previously introduced, the genetic testing data is curated and organized by specific categories. In addition to a category for identifying associated users, their genetic relationships, and their disease likelihoods, there is also a category that provides published thresholds based off scientific research for determining how serious a risk for a certain disease may be. Furthermore, the media guidance application may determine from the specific threshold category in the genetic testing data that the threshold value for Alzheimer's disease is 50%. As a result, the media guidance application may identify the son as exceeding the threshold.

At 720, the control circuitry 404 recommends disease-related content stored in memory 408 or media guidance data source 518 on the disease display 103, presented on display 412, to the selected second user that is determined to have a genetic link to the first user and that is associated with a likelihood of having a genetic link to the disease that exceeds the threshold. For example, the media guidance application may recommend Alzheimer's related content to the son. In a similar fashion to recommending content to the first user, the media guidance application may use a keyword search algorithm to search various forms of media assets that depict Alzheimer's disease. Additionally, the media guidance application may add additional search terms to the keyword search algorithm, such as son, father, prevention, etc. Once the media guidance application identifies the appropriate content to present to the first user, the media guidance application may populate media playlist 112 and program title column 114, program description column 116 and age rating column 118.

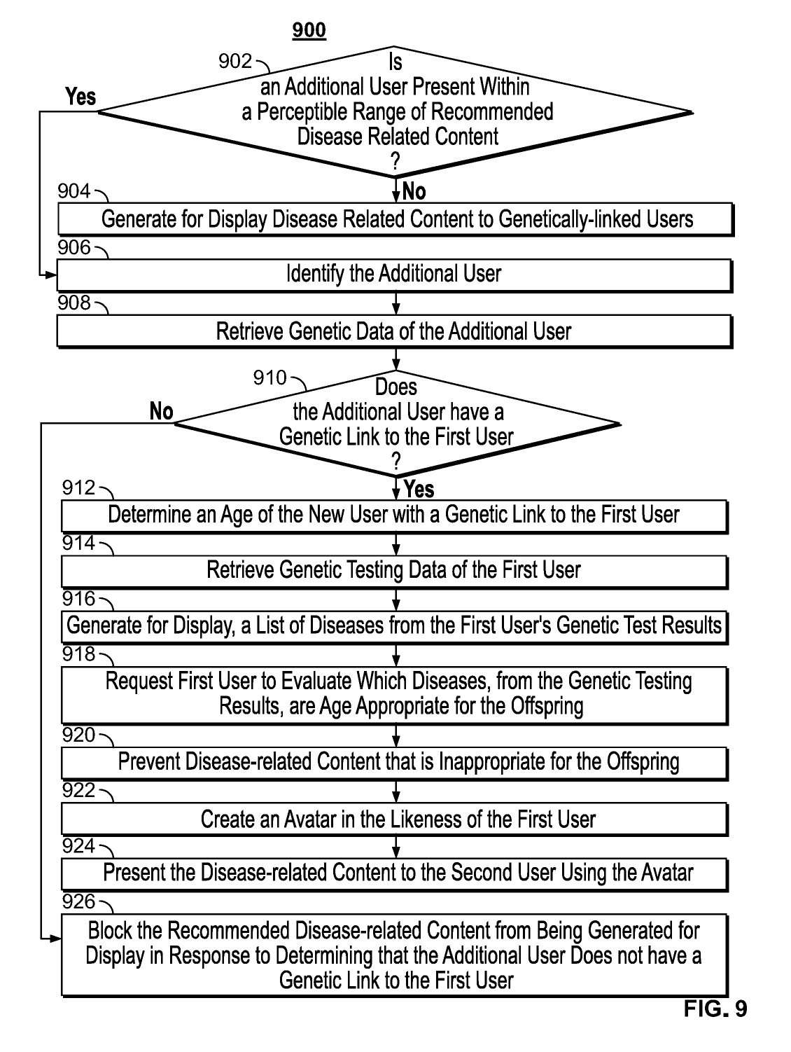

FIG. 8 is a flowchart of illustrative steps for generating a playlist of content based on a certain disease in accordance with some embodiments of the disclosure. It should be noted that process 800 or any step thereof could be performed on, or provided by, any of the devices shown in FIGS. 4-5. For example, process 800 may be executed by control circuitry 404 (FIG. 4) as instructed by a media guidance application implemented on a user device (e.g., user equipment devices 502, 504, and/or 506 (FIG. 5)) in order to generate a playlist of disease-based content. In addition, one or more steps of process 800 may be incorporated into or combined with one or more steps of any other process or embodiment (e.g., as described in relation to FIGS. 9-13)).

At 802, the control circuitry 404 retrieves a genetic sample from the first user. For example, the media guidance application may retrieve a genetic sample. In this case, “genetic sample” is a term that indicates a user submitted saliva to a testing entity and the media guidance application has received notification from the user of his or her submission of a genetic sample. For example, the first user may submit a genetic sample to 23andMe and concurrently the media guidance application may receive a notice that the first user has submitted a genetic sample.

At 804, the control circuitry 404 performs genetic analysis on the genetic sample of the first user to identify changes in at least one of the chromosomes, genes, and proteins. For example, the media guidance application retrieves, from a genetic testing provider such as 23andMe, the results of a genetic analysis. Since the media guidance application itself does not perform genetic testing on an actual genetic sample, the media guidance application may parse through the genetic testing data and retrieve relevant genetic analysis results.

At 806, the control circuitry 404 retrieves results of the first user based on the genetic analysis, which indicates at least one of a likelihood that the first user is a carrier of a particular genetic mutation and has an increased risk of developing a disease. For example, the media guidance application may retrieve relevant 23andMe results, as previously described, where a relevant result may be likelihood statistics for a user and the user's likelihood of being a carrier of a certain disease. For example, 23andMe may possesses an object data structure that is replicated in local memory when the media guidance application accesses genetic information from an entity, such as 23andMe. The media guidance application may access 23andMe's base object that contains a library of genetic testing data for multiple individuals. The media guidance application may, in a tree like fashion, access a group object that has the first user's genetic data. Furthermore, the media guidance application may access an object associated with the group object that has, attached to it, various categorical objects, such as likelihood values for associated diseases, related user data, blood data, etc. For example, the media guidance application may identify the first user as the father and extract data from the associated disease categorical object suggesting the first user has an increased risk of developing Alzheimer's disease.

At step 808, the control circuitry 404 determines whether the user has a social media presence. For example, the media guidance application may access the first user's profile and determine accompanying social network accounts. The media guidance application may determine the first user has Facebook, YouTube, and Twitter social network accounts.

At step 810, the control circuitry 404 searches a social network for users associated with the first user. For example, the media guidance application may identify, as previously described, the first user's various social media accounts. In doing so, the media guidance application may use a keyword associated algorithm that searches the user's social media outlets for keywords, such as Alzheimer's, disease, father, etc. The media guidance application may search all associated social networks and/or the media guidance application may receive commands from the user to search only a subset of the social networks in which the first user is associated. Additionally, the media guidance application may search for fathers with genetic test results that indicate a high likelihood of developing Alzheimer's disease.

At step 812, the control circuitry 404 generates a list of related users from the first user's social network. For example, the media guidance application may generate a list of Facebook users with the same Alzheimer's results.

At step 814, the control circuitry 404 searches a social network for user generated content associated with the disease. For example, the media guidance application may identify YouTube, which is a social media outlet dedicated to displaying videos, channels that are dedicated to educating fathers how to prepare for the challenges of an Alzheimer's diagnosis. In particular, YouTube channels are compilations of videos related to a certain theme.

At 816, the control circuitry 404 generates a playlist of content that includes a first media asset corresponding to a background of the first user based on the genetic data and a second media asset corresponding to the disease, based on the genetic data of the first user. For example, the media guidance application may retrieve the videos from the Alzheimer's disease preparation YouTube channel and present those videos in media playlist 112 (FIG. 1). The media guidance application may order the videos by relevance, based on a keyword algorithm search. Additionally, the media guidance application may organize the videos in media playlist 112 in chronological order. The media guidance application generates for display links in media playlist 112 to media assets that link to the source media asset file.

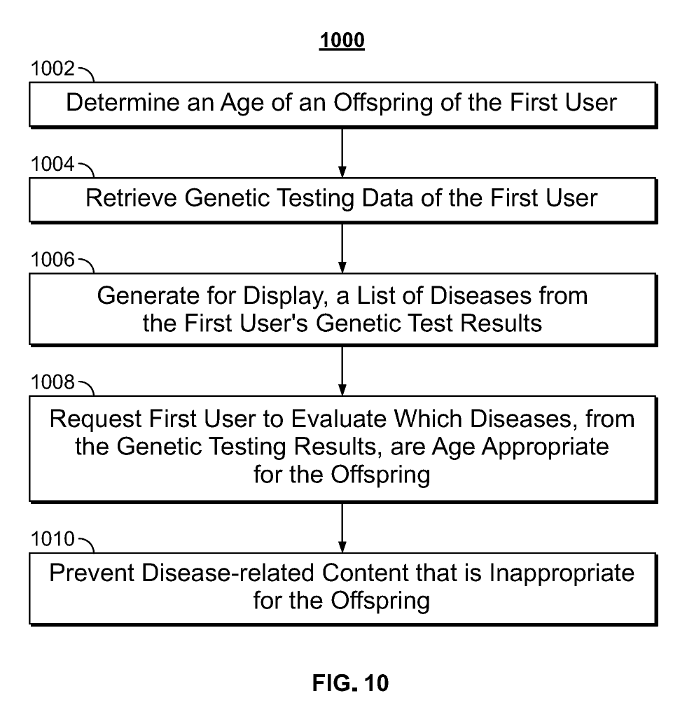

FIG. 9 is a flowchart of illustrative steps for blocking disease related-content to underage or non-genetically linked users in accordance with some embodiments of the disclosure. It should be noted that process 800 or any step thereof could be performed on, or provided by, any of the devices shown in FIGS. 4-5. For example, process 800 may be executed by control circuitry 404 (FIG. 4) as instructed by a media guidance application implemented on a user device (e.g., user equipment devices 502, 504, and/or 506 (FIG. 5)) in order to block disease related-content to underage or non-genetically-linked users. In addition, one or more steps of process 900 may be incorporated into or combined with one or more steps of any other process or embodiment (e.g., as described in relation to FIGS. 10-12)).

At 902, the control circuitry 404 determines whether an additional user is present within a perceptible range of recommended disease-related-content. For example, using facial recognition technology, the media guidance application may identify a new user in the viewing area of a television.

At 904, the control circuitry 404 generates for display disease-related content to genetically-linked users. For example, the media guidance application may determine no additional user is present in the viewing area and generate for display disease-related content to a first user's offspring.

At 906, the control circuitry 404 identifies the additional user. For example, the media guidance application may identify the additional user to be a friend of the son of the first user.

At 908, the control circuitry 404 retrieves genetic data of the additional user. For example, the media guidance application may request and retrieve the son's friend's genetic testing data from a genetic testing entity.

At 910, the control circuitry 404 determines whether the additional user has a genetic link to the first user. For example, the media guidance application may retrieve supplemental 23andMe data that indicates the son's friend does not have a genetic link to the first user. In an additional example, the media guidance application may identify the additional user to be the son. In this case, the media guidance application may determine the son to have a genetic link to the first user, the father. For additional detail, refer to previously described processes and techniques in FIG. 7-8.

At 912, the control circuitry 404 determines the age of the new user with a genetic link to the first user. For example, in the case of the son, the media guidance application may determine the son to be eight years old. The media guidance application, as previously described, may use facial recognition, voice recognition, or may access profile information that indicates the new user's age.

At 914, the control circuitry 404 retrieves genetic testing data of the first user. For example, the media guidance application may retrieve the father's genetic testing data. Refer to FIG. 7 for more detail on receiving genetic testing data.

At 916, the control circuitry 404 generates for display, a list of diseases from the first user's genetic test results. For example, the media guidance application may retrieve from the father's genetic testing data a list of diseases that the father has a high likelihood of developing. Refer to FIG. 7-8 for more detail on generating lists of diseases.

At 918, the control circuitry 404 requests the first user to evaluate which diseases, from the genetic testing results, are age-appropriate for the offspring. For example, the media guidance application may receive input from the first user designating which diseases from the list of diseases are age appropriate for the eight-year-old son.

At 920, the control circuitry 404 prevents disease-related content that is inappropriate for the offspring. For example, the media guidance application may prevent the generating for display of content deemed, by the father, to be inappropriate for the son. The media guidance application may block content by assigning an identifier to inappropriate content. Once the inappropriate content is assigned with an identifier, the media guidance application may restrict generating for display that content.

At 922, the control circuitry 404 creates avatar 110 in the likeness of the first user. For example, the media guidance application may use 3D animation technology to develop an avatar of the father. The media guidance application may denote sex (male or female), face shape, eye color, hair shape and color, and/or preferred clothing style. The media guidance application may then store the avatar in local and/or remote memory, which can be accessed and displayed in disease display 103.

At 924, the control circuitry 404 presents the disease-related content to the second user using avatar 110. For example, the media guidance application may use avatar 110 as a conduit to communicate information to the second user (e.g., the son). For example, the media guidance application may record the father giving commentary on Alzheimer's and why it is important to learn more about the disease. The media guidance application may also record common catchphrases of the father to make the son more comfortable when navigating through disease-related content.

At 926, the control circuitry 404 blocks the recommended disease-related content from being generated for display in response to determining that the additional user does not have a genetic link to the first user. For example, the media guidance application may determine, based off previous steps, the content is either age-inappropriate for the second user or the additional user is not genetically linked to the first user and block content.

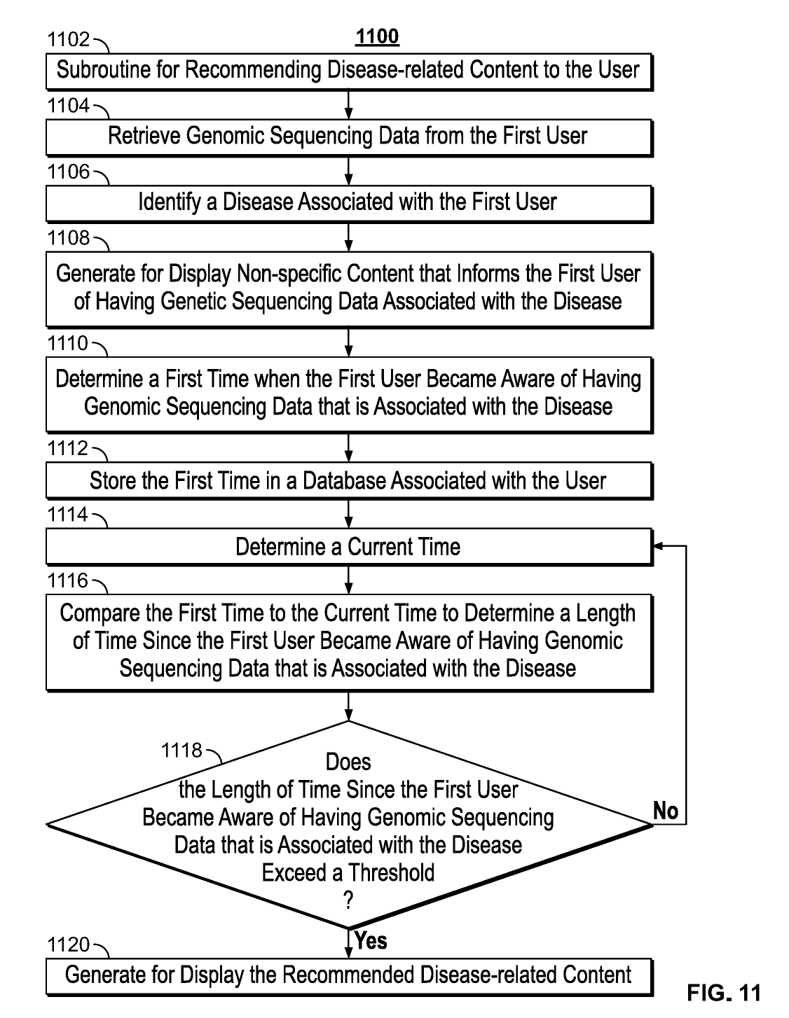

FIG. 10 is a flowchart of illustrative steps for preventing age-inappropriate disease related content in accordance with some embodiments of the disclosure. It should be noted that process 1000 or any step thereof could be performed on, or provided by, any of the devices shown in FIGS. 4-5. For example, process 800 may be executed by control circuitry 404 (FIG. 4) as instructed by a media guidance application implemented on a user device (e.g., user equipment devices 502, 504, and/or 506 (FIG. 5)) in order to prevent age-inappropriate disease related content. In addition, one or more steps of process 900 may be incorporated into or combined with one or more steps of any other process or embodiment (e.g., as described in relation to FIGS. 11-13).

At 1002, control circuitry 404 determines an age of an offspring of the first user. For example, the media guidance application may access user profiles that contain age related information and may determine the first user, the father, has a daughter who is twelve years old.

At 1004, control circuitry 404 retrieves genetic testing data of the first user. For example, the media guidance application may retrieve the father's 23andMe genetic testing data. Refer to FIG. 7-8 for further description on retrieving testing data.

At 1006, control circuitry 404 generates for display a list of diseases from the first user's genetic test results. For example, the media guidance application may retrieve a list of diseases associated with the first user. Refer to FIG. 7-8 for further description on generating a list of diseases.

At 1008, control circuitry 404 requests the first user to evaluate which diseases, from the genetic testing results, are age-appropriate for the offspring. For example, the media guidance application may receive input from the father designating which programs are appropriate for the daughter. Refer to FIG. 9 for more details.

At 1010, control circuitry 404 prevents disease-related content that is inappropriate for the offspring. For example, the father may have deemed a live surgery to be too graphic for the daughter and prevent any surgery-related content to be generated for display. Refer to FIG. 9 for more details.

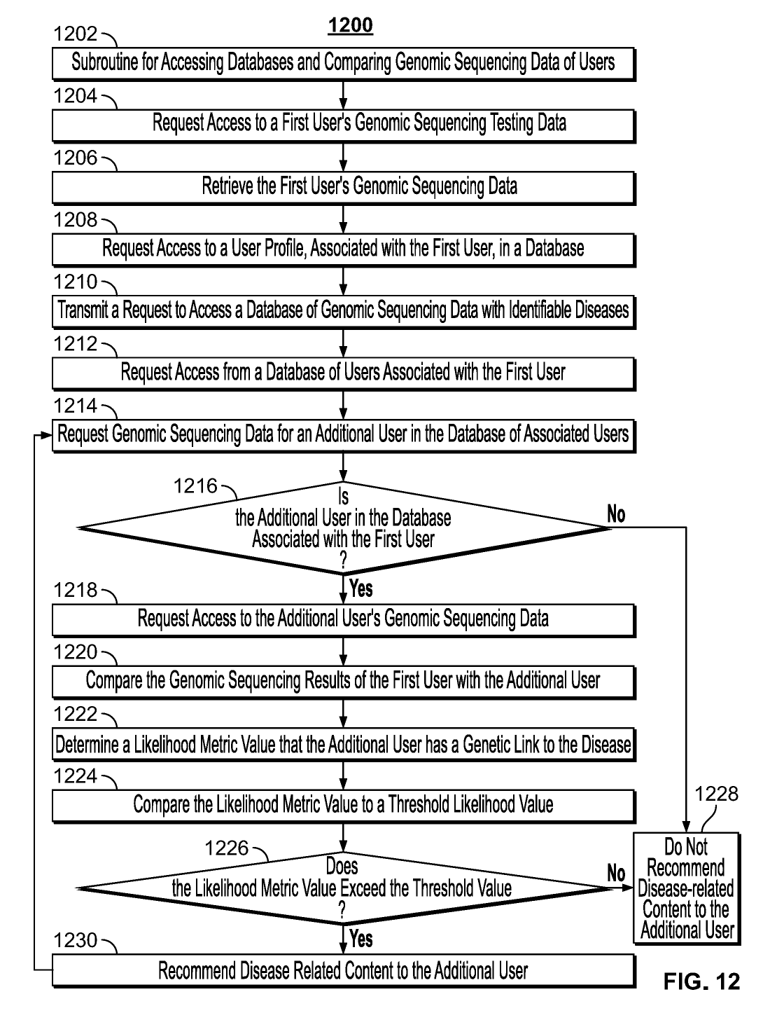

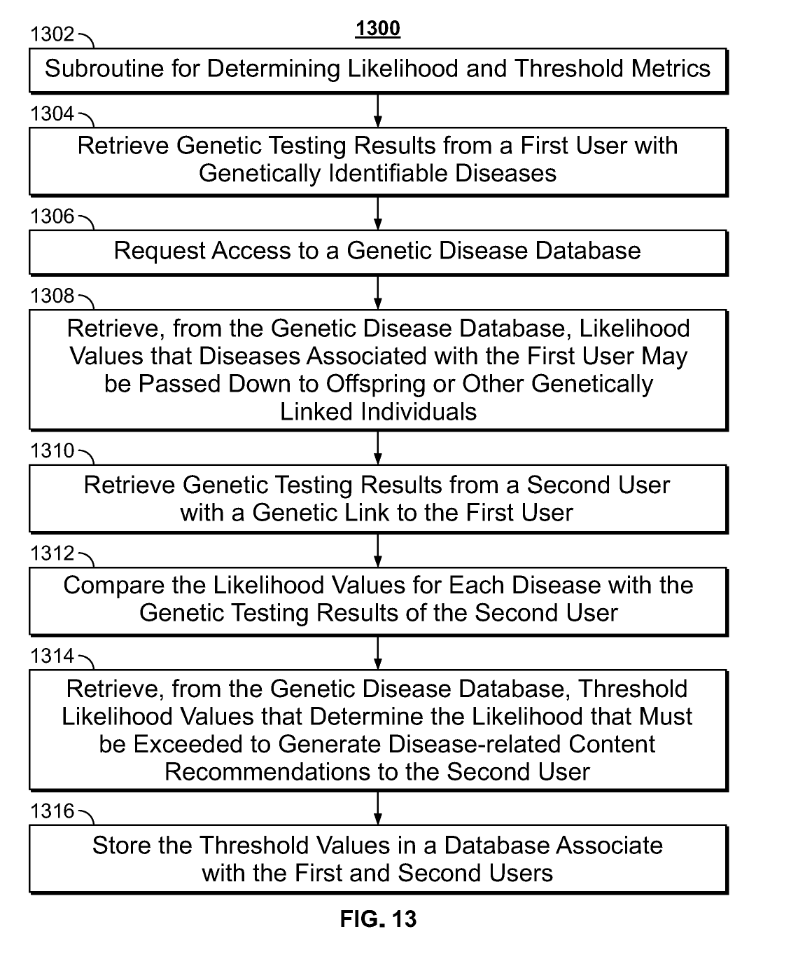

FIG. 11 is a flowchart of illustrative steps for recommending disease related content to the user in accordance with some embodiments of the disclosure. It should be noted that process 1100 or any step thereof could be performed on, or provided by, any of the devices shown in FIGS. 4-5. For example, process 1100 may be executed by control circuitry 404 (FIG. 4) as instructed by a media guidance application implemented on a user device (e.g., user equipment devices 502, 504, and/or 506 (FIG. 5)) in order to recommend disease related content to the user. In addition, one or more steps of process 1100 may be incorporated into or combined with one or more steps of any other process or embodiment (e.g., as described in relation to FIG. 12-13)).

At 1102, control circuitry 404 begins a subroutine for recommending disease-related content to the user. For example, the media guidance application may recommend disease related content to a user, displayed in media playlist 112 in disease display 103. The media guidance application may parse through a media asset database both locally and/or remotely using keyword algorithm searches for words related to the disease or related key terms.

At 1104, control circuitry 404 retrieves genomic sequencing data for the first user. For example, the media guidance application may retrieve the father's genomic testing data from 23andMe. Refer to FIG. 7-8 for more detail.

At 1106, control circuitry 404 identifies a disease associated with the first user. For example, the media guidance application may determine from the company data, such as 23andMe, that the father has an increased likelihood of developing Alzheimer's disease. Refer to FIGS. 7-8 for more detail.

At 1108, control circuitry 404 generates for display non-specific content that informs the first user of having genetic sequencing data associated with the disease, where “non-specific content” means no specific diseases are mentioned. For example, the media guidance application may generate for display a message in disease display 103 that informs the first user that genetic testing data is available that is associated with a disease.

At 1110, control circuitry 404 determines a first time when the first user became aware of having genomic sequencing data that is associated with the disease. For example, the media guidance application may determine a time in which the first user was first made aware that disease-related genetic testing data is available. The media guidance application may recognize the first user was made aware by the following techniques, facial recognition, manual entry on a remote device in response to a prompt, auditory sensors to gauge any elevated tones, a heart rate monitor that can detect increased stress levels after viewing new or potentially life-altering information, etc.

At 1112 control circuitry 404 stores the first time in a database associated with the user. For example, the media guidance application may store the first time in local memory on the user's profile. The media guidance application may store the time as an object in an object data structure, as previously described, where the first-time object is a categorical object.

At 1114, control circuitry 404 determines a current time. For example, the media guidance application may use an internal clock, within the media guidance application, to determine the current time. The media guidance application may store the current time temporarily in local memory in a temporary object that can be changed and updated.

At 1116, control circuitry 404 compares the first time to the current time to determine a length of time since the first user became aware of having genomic sequencing data that is associated with the disease. For example, the media guidance application may determine the difference in time between the two time stamps is one week. The media guidance application may have functionality to subtract the time values to gain a time difference.

At 1118, control circuitry 404 determines whether the length of time since the first user became aware of having genomic sequencing data that is associated with the disease exceeds a threshold. For example, the media guidance application may determine based off user preference the threshold time to be three days. Furthermore, the media guidance application may determine that the difference in time as previously explained, one week, exceeds the threshold.

At 1120, control circuitry 404 generates for display the recommended disease related content to the first user. For example, the media guidance application may generate for display Alzheimer's disease-related content for the first user in media playlist 112 of disease display 103.

FIG. 12 is a flowchart of illustrative steps for accessing genomic sequencing databases in accordance with some embodiments of the disclosure. It should be noted that process 1200 or any step thereof could be performed on, or provided by, any of the devices shown in FIGS. 4-5. For example, process 1200 may be executed by control circuitry 404 (FIG. 4) as instructed by a media guidance application implemented on a user device (e.g., user equipment devices 502, 504, and/or 506 (FIG. 5)) in order to access genomic sequencing databases. In addition, one or more steps of process 1100 may be incorporated into or combined with one or more steps of any other process or embodiment (e.g., as described in relation to FIG. 13)).

At 1202, control circuitry 404 begins a subroutine for accessing databases and comparing genomic sequencing data of users. For example, the media guidance application may access and retrieve genetic testing data for a variety of users.

At 1204, control circuitry 404 requests access to a first user's genomic sequencing testing data. For example, the media guidance application may request access to the father's 23andMe genetic testing data. Refer to FIG. 7-8 for more detail.

At 1206, control circuitry 404 retrieves the first user's genomic sequencing data. For example, the media guidance application may retrieve the father's 23andMe data. Refer to FIGS. 7-8 for more detail.

At 1208, control circuitry 404 requests access to a user profile, associated with the first user, in a database stored locally and/or remotely. For example, the media guidance application may request access to the 23andMe profile for the father. Refer to FIGS. 7-8 for more detail.

At 1210, control circuitry 404 transmits a request to access a database of genomic sequencing data with identifiable diseases. For example, the media guidance application may request access to the 23andMe database of sequencing data and diseases based off the father's results.

At 1212, control circuitry 404 requests access from a database stored locally and/or remotely of users associated with the first user. For example, the media guidance application may request access from several users that are on an associated local profile, as previously described. For example, the media guidance application may request access to the mother, daughter, and son.

At 1214, control circuitry 404 requests genomic sequencing data for an additional user in the database of associated users stored locally and/or remotely. For example, as previously explained, the media guidance application may request the son's genetic sequencing data. Refer to FIGS. 7-8 for more detail in requesting genetic sequencing data.

At 1216, control circuitry 404 determines if the additional user in the database stored locally and/or remotely is associated with the first user. For example, the media guidance application may determine if the son is associated with the father. Refer to FIGS. 7-8 for more detail on associated users.

At 1218, control circuitry 404 requests access to the additional user's genomic sequencing data. For example, the media guidance application may request access to son's 23andMe genetic testing data. Refer to FIGS. 7-8 for more detail for accessing genetic sequencing data.

At 1220, control circuitry 404 compares the genomic sequencing results of the first user with the additional user. For example, the media guidance application may retrieve the genetic testing data for the father and son and, based off the results and analysis from the genetic testing database, compare the two users. Furthermore, as previously mentioned, the media guidance application may not perform analytic genetic testing. However, the media guidance application may retrieve genetic testing data, arranged in an object-based data structure, and utilize the categorical objects to make determinations from the genetic data.

At 1222, control circuitry 404 determines a likelihood metric value that the additional user has a genetic link to the disease. For example, the media guidance application may retrieve from the 23andMe directory metric data that indicates whether a genetic link to the disease exists between the father and the son. Refer to data structure explanations in FIGS. 7-8.

At 1224, control circuitry 404 compares the likelihood metric value to a threshold likelihood value. For example, the media guidance application may determine from the 23andMe analysis, comparison results between the father and the son that compare the likelihood values with the threshold likelihood values. Refer to FIGS. 7-8 for more details.

At 1226, control circuitry 404 determines if the likelihood metric value exceeds the threshold value. For example, the media guidance application may determine the likelihood metric value from the 23andMe results exceeds the threshold, meaning Alzheimer's disease is a risk for the genetically-linked son, too. Additionally, the media guidance application may have a subtraction function built in that can assess the difference between values and temporarily store those values in a categorical storage object locally and/or remotely.

At 1228, control circuitry 404 does not recommend disease-related content to the additional user. For example, the media guidance application may determine, based off a likelihood comparison, that the son does not have an elevated risk of developing Alzheimer's disease. As a result, the media guidance application may not recommend Alzheimer's disease-related content to the son in media playlist 112 on disease display 103.

At 1230, control circuitry 404 recommends disease related content to the additional user. For example, the media guidance application may recommend Alzheimer's-related content to the son.

FIG. 13 is a flowchart of illustrative steps for determining disease likelihood and threshold metrics in accordance with some embodiments of the disclosure. It should be noted that process 1300 or any step thereof could be performed on, or provided by, any of the devices shown in FIGS. 4-5. For example, process 1200 may be executed by control circuitry 404 (FIG. 4) as instructed by a media guidance application implemented on a user device (e.g., user equipment devices 502, 504, and/or 506 (FIG. 5)) in order to determine disease likelihood and threshold metrics. In addition, one or more steps of process 1100 may be incorporated into or combined with one or more steps of any other process or embodiment.

At 1302, control circuitry 404 begins a subroutine for determining likelihood and threshold metrics. For example, the media guidance application may retrieve likelihood and threshold metric data from a genetic testing service's database, such as 23andMe. Refer to FIGS. 7-8 for more detail.

At 1304, control circuitry 404 retrieves genetic testing results from a first user with genetically identifiable diseases. For example, the media guidance application may retrieve the father's genetic testing results from 23andMe. Refer to FIGS. 7-8 for more detail.

At 1306, control circuitry 404 requests access to a genetic disease database. For example, the media guidance application may request access to the 23andMe database. Refer to FIG. 7-8 for more detail.

At 1308, control circuitry 404 retrieves, from the genetic disease database, likelihood values that diseases associated with the first user may be passed down to offspring or other genetically linked individuals. For example, the media guidance application may retrieve supplemental data that suggests likelihood values for passing down certain genetic diseases to offspring. Refer to FIG. 7-8 for more detail and commentary on data structures.

At 1310, control circuitry 404 retrieves genetic testing results from a second user with a genetic link to the first user. For example, the media guidance application may retrieve the son's 23andMe genetic testing data.

At 1312, control circuitry 404 compares the likelihood values for each disease with the genetic testing results of the second user. For example, the media guidance application may retrieve supplemental data from the entity's directory and use that data to retrieve comparative results of disease likelihoods in the second user. Refer to FIG. 7-8 for more detail.

At 1314, control circuitry 404 retrieves, from the genetic disease database, threshold likelihood values that determine the likelihood that must be exceeded to generate disease-related content recommendations to the second user. For example, the media guidance application may assess, based off the 23andMe data, whether the son's likelihood of having certain diseases exceeds a threshold. Refer to FIG. 11 for more detail on comparison techniques.

At 1316, control circuitry 404 stores the threshold values associated with the first and second users in a database stored locally and/or remotely. For example, the media guidance application may retrieve and store the threshold values found in the 23andMe database to easily access the values for analysis. Refer to FIGS. 7-8 for more detail for storage and data structure explanation.

It should be noted that processes 600-1300 or any step thereof could be performed on, or provided by, any of the devices shows in FIGS. 1 and 4-5. For example, any of processes 600-1200 may be executed by control circuitry 404 (FIG. 4) as instructed by control circuitry implemented on user equipment 502, 504, 506 (FIG. 5), and/or a user equipment for selecting a recommendation. In addition, one or more steps of processes 600-1300 may be incorporated into or combined with one or more steps of any other process or embodiment.

It is contemplated that the steps or descriptions of each of FIGS. 6-13 may be used with any other embodiment of this disclosure. In addition, the steps and descriptions described in relation to FIGS. 6-13 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 1 and 4-5 could be used to perform one or more of the steps in FIGS. 6-13.

It will be apparent to those of ordinary skill in the art that methods involved in the present invention may be embodied in a computer program product that includes a computer-usable and/or readable medium. For example, such a computer-usable medium may consist of a read-only memory device, such as a CD-ROM disk or conventional ROM device, or a random access memory, such as a hard drive device or a computer diskette, having a computer-readable program code stored thereon. It should also be understood that methods, techniques, and processes involved in the present disclosure may be executed using processing circuitry. For instance, blacking out in results display 102 may be performed, e.g., by processing circuitry 406 of FIG. 4. The processing circuitry, for instance, may be a general purpose processor, a customized integrated circuit (e.g., an ASIC), or a field-programmable gate array (FPGA) within user equipment 400, media content source 516, or media guidance data source 518. For example, a profile, as described herein, may be stored in, and retrieved from, storage 408 of FIG. 4, or media guidance data source 518 of FIG. 5. Furthermore, processing circuitry, or a computer program, may update settings of the tournament-style competition results display 102, stored within storage 408 of FIG. 4 or media guidance data source 518 of FIG. 5.

The processes discussed above are intended to be illustrative and not limiting. One skilled in the art would appreciate that the steps of the processes discussed herein may be omitted, modified, combined, and/or rearranged, and any additional steps may be performed without departing from the scope of the invention. More generally, the above disclosure is meant to be exemplary and not limiting. Only the claims that follow are meant to set bounds as to what the present invention includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real time. It should also be noted, the systems and/or methods described above may be applied to, or used in accordance with, other systems and/or methods.

While some portions of this disclosure may make reference to “convention,” any such reference is merely for the purpose of providing context to the invention(s) of the instant disclosure, and does not form any admission as to what constitutes the state of the art.

1. (canceled)

2. A method for recommending content, featuring a genetically identifiable disease, based on a positive determination of a genetic marker that suggests a likelihood of having a certain disease, the method comprising:
requesting access to a first user's genomic sequencing testing data;
in response to requesting the first user's genomic sequencing testing data, retrieving from a profile associated with the first user, the first user's genomic sequencing testing data;
searching, based on the retrieved first user's genomic sequencing testing data, a database that associates genomic sequencing data with diseases to identify a disease associated with the first user;
accessing a list of users associated with the first user;
comparing genetic data of each user in the list of users with genetic data of the first user;
determining, based on the comparing, a subset of the list of users that has a genetic link to the first user;
determining for each given user in the subset a likelihood that the given user in the subset has a genetic link to the disease based on the genetic data associated with the given user in the subset;
in response to determining the likelihood of each user in the subset, selecting a second user within the subset that is associated with a likelihood that exceeds a threshold; and
recommending disease-related content to the selected second user that is determined to have a genetic link to the first user and that is associated with a likelihood of having a genetic link to the disease that exceeds the threshold.

3. The method of claim 2, wherein the genetic-related disease comprises:
a disorder or condition caused by abnormalities in a person's genome;
wherein a genome further comprises genetic material of an organism, such as a human, that contains information about that organism's traits and characteristics, including the organism's likelihood of having a certain disease.

4. The method of claim 2, wherein requesting access to a first user's genomic sequencing testing data comprises:
retrieving a genetic sample from the first user;
performing genetic analysis on the genetic sample of the first user to identify changes in at least one of the chromosomes, genes, and proteins;
presenting results to the first user based on the analysis, which indicates at least one of a likelihood that the first user is a carrier of a particular genetic mutation, the first user is associated with an increased risk of developing a disease, such as cancer, in the future, and a need for further testing.

5. The method of claim 2, further comprising:
searching a social network for user generated content associated with the disease; and
causing the user generated content to be presented to the first and second users as the recommendation.

6. The method of claim 2, further comprising generating content for presentation to the first user based on the genetic data of the first user.

7. The method of claim 2 further comprising generating a playlist of content that includes a first media asset corresponding to a background of the first user based on the genetic data and a second media asset corresponding to the disease.

8. The method of claim 2 further comprising:
determining an age of an offspring of the first user;
retrieving genetic testing data of the first user;
generating for display, a list of diseases from the first user's genetic test results;
requesting first user to evaluate which diseases, from the genetic testing results, are age appropriate for the offspring; and
based off the first user's evaluation of appropriate content based on offspring age, preventing disease-related content that is inappropriate for the offspring.

9. The method of claim 2 further comprising:
determining whether an additional user is within a perceptible range of the recommended disease-related content;
determining an identity of the additional user;
retrieving genetic data of the additional user;
determining, based on the genetic data, whether the additional user has a genetic link to the first user; and
blocking the recommended disease-related content from being generated for display in response to determining that the additional user does not have a genetic link to the first user.

10. The method of claim 2 further comprising:
determining a first time when the first user became aware of having genomic sequencing data that is associated with the disease;
comparing the first time to a current time to determine a length of time since the first user became aware of having genomic sequencing data that is associated with the disease; and
in response to determining that the length of time exceeds a threshold, generating for display the recommended disease-related content.

11. The method of claim 2 further comprising:
creating an avatar in the likeness of the first user; and
presenting the disease-related content to the second user using the avatar.

12. A system for recommending content, featuring a genetically identifiable disease, based on a positive determination of a genetic marker that suggests a likelihood of having a certain disease, the system comprising:
communications circuitry; and
control circuitry configured to:
request access to a first user's genomic sequencing testing data;
in response to requesting the first user's genomic sequencing testing data, retrieve from a profile associated with the first user, the first user's genomic sequencing testing data;
search, based on the retrieved first user's genomic sequencing testing data, a database that associates genomic sequencing data with diseases to identify a disease associated with the first user;
access a list of users associated with the first user;
compare genetic data of each user in the list of users with genetic data of the first user;
determine, based on the comparing, a subset of the list of users that has a genetic link to the first user;
determine for each given user in the subset a likelihood that the given user in the subset has a genetic link to the disease based on the genetic data associated with the given user in the subset;
in response to determining the likelihood of each user in the subset, select a second user within the subset that is associated with a likelihood that exceeds a threshold; and
recommend disease-related content to the selected second user that is determined to have a genetic link to the first user and that is associated with a likelihood of having a genetic link to the disease that exceeds the threshold.

13. The system of claim 12, wherein the genetic-related disease comprises:
a disorder or condition caused by abnormalities in a person's genome; and
wherein a genome further comprises genetic material of an organism, such as a human, that contains information about that organism's traits and characteristics, including the organism's likelihood of having a certain disease.

14. The system of claim 12, wherein the control circuitry configured to request access to a first user's genomic sequencing testing data is further configured to:
retrieve a genetic sample from the first user;
perform genetic analysis on the genetic sample of the first user to identify changes in at least one of the chromosomes, genes, and proteins; and
present results to the first user based on the analysis, which indicates at least one of a likelihood that the first user is a carrier of a particular genetic mutation, the first user is associated with an increased risk of developing a disease, such as cancer, in the future, and a need for further testing.

15. The system of claim 12, wherein the control circuitry is further configured to:
search a social network for user generated content associated with the disease; and
cause the user generated content to be presented to the first and second users as the recommendation.

16. The system of claim 12, wherein the control circuitry is further configured to generate content for presentation to the first user based on the genetic data of the first user.

17. The system of claim 12, wherein the control circuitry is further configured to generate a playlist of content that includes a first media asset corresponding to a background of the first user based on the genetic data and a second media asset corresponding to the disease.

18. The system of claim 12, wherein the control circuitry is further configured to:
determine an age of an offspring of the first user;
retrieve genetic testing data of the first user;
generate for display, a list of diseases from the first user's genetic test results;
request first user to evaluate which diseases, from the genetic testing results, are age appropriate for the offspring; and
based off the first user's evaluation of appropriate content based on offspring age, prevent disease-related content that is inappropriate for the offspring from being presented.

19. The system of claim 2, wherein the control circuitry is further configured to:
determine whether an additional user is within a perceptible range of the recommended disease-related content;
determine an identity of the additional user;
retrieve genetic data of the additional user;
determine, based on the genetic data, whether the additional user has a genetic link to the first user; and
block the recommended disease-related content from being generated for display in response to determining that the additional user does not have a genetic link to the first user.

20. The system of claim 12, wherein the control circuitry is further configured to:
determine a first time when the first user became aware of having genomic sequencing data that is associated with the disease;
compare the first time to a current time to determine a length of time since the first user became aware of having genomic sequencing data that is associated with the disease; and
in response to determining that the length of time exceeds a threshold, generate for display the recommended disease-related content.

21. The system of claim 12, wherein the control circuitry is further configured to:
create an avatar in the likeness of the first user; and
present the disease-related content to the second user using the avatar.

22-51. (canceled)
